(19) United States
(12) Patent Application Publication
(10) Pub. No.: US 2022/0378307 A1
(43) Pub. Date: Dec. 1, 2022

(54) METHOD FOR CORRECTING PULSE WAVETRANSIT TIME ASSOCIATED WITH DIASTOLIC BLOOD PRESSURE OR SYSTOLIC BLOOD PRESSURE

(71) Applicant: ZHEJIANG MAILIAN MEDICAL EQUIPMENT CO., LTD, HANGZHOU (CN)

(72) Inventors: YAN CHEN, HANGZHOU (CN); YU CHEN, HANGZHOU (CN)

(73) Assignee: ZHEJIANG MAILIAN MEDICAL EQUIPMENT CO., LTD, HANGZHOU (CN)

(21) Appl. No.: 17/874,618

(22) Filed: Jul. 27, 2022

Related U.S. Application Data
(63) Continuation-in-part of application No. 16/391,287, filed on Apr. 22, 2019.
Application No. 16/391,287 is a continuation of application No. PCT/CN2017/111799, filed on Nov. 20, 2017.

(30) Foreign Application Priority Data
Nov. 22, 2016 (CN) 201611045054.5
Nov. 22, 2016 (CN) 201611046184.0

(51) Int. Cl. A61B 5/021; A61B 5/0285

(52) U.S. Cl. CPC A61B 5/02125; A61B 5/0285; A61B 5/02116

(57) ABSTRACT

The present invention relates to a method for correcting pulse wave transit time associated with diastolic blood pressure and systolic blood pressure, and the correction method can perform adaptive correction of the irregular change of pulse wave transit time caused by blood transfusion and intravenous transfusion, vasoactive drugs, surgical intervention, etc. in a clinical setting. A pulse wave transit time is determined by a time difference of an ear pulse wave and a toe pulse wave in the same cardiac cycle, and a few correction variables are extracted based on the pulse wave features, then a total correction value is acquired to perform correction on the irregular change of pulse wave transit time. The corrected transit time can be used with available mathematical models for continuously measuring diastolic blood pressure and systolic blood pressure in each cardiac cycle in a clinical setting with high accuracy.

### CROSS REFERENCE TO RELATED PATENT APPLICATIONS

The present application is a continuation in part application of the U.S. Ser. No. 16/391,287 filed Apr. 22, 2019, which is the PCT/CN2017/111799 enters US national stage, claims the priorities of CN 201611046184.0 filed Nov. 22, 2016 and CN 201611045054.5 filed Nov. 22, 2016, above-mentioned applications are incorporated herein by reference.

### BACKGROUND OF THE INVENTION

### 1. Field of the Invention

The present invention relates to the technical field of arterial blood pressure measurement, and specifically relates to a method for correcting pulse wave transit time associated with diastolic blood pressure or systolic blood pressure.

### 2. Description of Related Art

Arterial blood pressure is one of the main indicators for reflecting the state of a circulatory system and assessing organ perfusion, and is an important vital sign parameter for perioperative monitoring. At present, the methods of perioperative blood pressure monitoring can be divided into invasive measurement and non-invasive measurement. Invasive measurement refers to a technique of placing a catheter into artery, and converting Intra-arterial pressure into an electronic signal through a transducer, and display the blood pressure signal in real time on a monitoring device. Invasive method can measure beat-to-beat blood pressure continuously and accurately, but the possible dangers and injuries cannot be ignored. Oscillometric method is commonly used for non-invasive measurement, which is simple to operate, is clinically recognized in accuracy and is widely used for health check-up and perioperative monitoring. However, the oscillometric method only measures blood pressure intermittently in every 3-5 minutes intraoperatively, and cannot track changes in arterial blood pressure in real time.

To this end, some techniques for continuous and non-invasive measurement of beat-to-beat blood pressure have been proposed, in which the measurement method based on pulse wave transit time/velocity (PTT/PWV) has gradually become a research hotspot. The method is to simultaneously acquire photoplethysmography (PPG) signals and an electrocardiogram (ECG) by one or more photosensors and electrocardiographic electrodes, and PTT/PWV is calculated by using the time difference between a PPG and an ECG or the time difference between two PPGs; Afunctional relationship or mathematical model between PTT/PWV and blood pressure is established, so that the measurable PTT/PWV can be used for calculating blood pressure. Some academic papers have reported the principle of continuous and non-invasive measurement of beat-to-beat blood pressure using PTT/PWV, for example, Yan Chen, Changyun Wen, Guocai Tao, Min Bi, and Guoqi Li A Novel Modeling Methodology of the Relationship Between Blood Pressure and Pulse Wave Velocity; Yan Chen, Changyun Wen, Guocai Tao and Min Bi Continuous and Non-invasive Measurement of Systolic and Diastolic Blood Pressure by One Mathematical Model with the Same Model Parameters and Two Separate Pulse Wave Velocities; Younhee Choi, Qiao Zhang, Seokbum Ko Non-invasive cuffless blood pressure estimation using pulse transit time and Hilbert-Huang transform; Zheng Y, Poon C C, Yan B P, Lau J Y Pulse Arrival Time Based Cuff-Less and 24-H Wearable Blood Pressure Monitoring and its Diagnostic Value in Hypertension; Mukkamala R, Hahn J O, Ivan O T, Mestha L K, Kim C S, Töreyin H, Kyal S Toward Ubiquitous Blood Pressure Monitoring via Pulse Transit Time: Theory and Practice. Moreover, some patents disclose specific methods or devices for continuous and non-invasive measurement of beat-to-beat blood pressure using PTT/PWV, for example, Chinese patents CN101229058A, CN102811659A and CN1127939C, U.S. Pat. Nos. 5,865,755, 5,857,975, 5,649,543 and 9,364,158, and European Patent 0413267, etc.

Existing methods and techniques for measuring blood pressure using PTT/PWV require conventional auscultatory or oscillometric method to measure one or more blood pressure values for initial calibration. The reason for the calibration is that the relationship between PTT/PWV and blood pressure is object-dependent, that is, there is a definite relationship between PTT/PWV and blood pressure of each individual. The purpose of calibration is to determine mathematical model parameters that are adaptive to the object.

However, the existing methods have certain limitations and can only be applied under the condition that the circulatory system is not subjected to external interference, because only in the absence of interference, the relationship between PTT and blood pressure has strong regularity for individuals, and can be described by certain functions and mathematical models. However, during the perioperative period, the circulatory system of a patient is influenced by fluid therapy, drugs, surgical operations, temperature, etc., which leads to a series of irregular changes of PTT. Use of an irregular PTT and an intrinsic mathematical model to calculate blood pressure may produce large errors. Because the relationship between irregular PTT and blood pressure no longer has certain regularity, even if the mathematical model parameters are frequently calibrated to adapt to the PTT irregularities, the fundamental problem is not solved, and consequently, the accuracy and realtime performance of existing methods cannot meet the requirement of clinical blood pressure measurement.

[symbol] Chen Yan. Continuous and Noninvasive Blood Pressure Measurement by Pulse Wave Velocity: a New and Systematic Modeling Methodology, Singapore, Nanyang Technological University, 2012

[symbol]

In the above thesis published by the inventor earlier, the main content of thisthesis is to describe the construction of the measuring device, the method of identifying Td and Ts, and how to use the relationship between blood pressure and PTT (mathematical model) to calculate the blood pressure value per heartbeat. It is worth noting that: Td and Ts mentioned in the thesis are not corrected, which can be used on normal people for measuring blood pressure. However, the uncorrected Td and Tsare not suitable for using in clinical environments, especially surgical environments. This is because the use of anesthetics and vasoactive drugs, blood transfusion, surgical intervention and other factors can cause abnormal changes in Td and Ts. The Td and Ts that contain abnormal changes will deviate from the original mathematical model, resulting in a large error in the calculated blood pressure.

### SUMMARY OF THE INVENTION

In view of deficiencies in the prior art, the present invention is directed to provide a method for correcting pulse wave transit time (PTT) associated with diastolic blood pressure, and the method can perform adaptive correction of the irregular changes of PTT caused by blood transfusion and intravenous (IV) transfusion, vasoactive drugs, surgical intervention, etc. with high accuracy in a clinical setting.

The present invention is further directed to provide a method for correcting PTT associated with systolic blood pressure, and the method can perform adaptive correction of the irregular changes of PTT caused by blood transfusion and IV transfusion, vasoactive drugs, surgical intervention, etc. with high accuracy in a clinical setting.

The pulse wave transit time corrected by this method can be used in various mathematical models which relate pulse wave transit time to blood pressure. In fact, studying the relationship between pulse wave transit time and blood pressure is another independent technology. There have been many papers that have published the mathematical model between pulse wave transit time and blood pressure (see the following references). The method provided by the present invention can be used in these mathematical models for real-timely correcting the pulse wave transit time, and then obtaining a more accurate blood pressure value in real clinical environment.

### REFERENCES

(1) Fung, P., G. Dumont, C. Ries, C. Mott, and M. Ansermino Continuous noninvasive blood pressure measurement by pulse transit time. The 26th Annual International Conference of the IEEE EMBS, San Francisco, Calif., 2004

In this paper, the mathematical model between blood pressure BP and pulse wave transit time PPT is described in equation (5)

$$\begin{matrix}BP = Δ BP / 0.7 \\ = \frac{1}{0.7} ( \frac{1}{2} ρ \frac{{d}^{2}}{{PTT}^{2}} + ρ gh ) \\ = \frac{A}{{PTT}^{2}} + B\end{matrix} \qquad ( 5 )$$

And formula (6) describes how to obtain the model coefficient A

$$A = {( 0.6 \times height )}^{2} \times \frac{p}{1.4} \qquad ( 6 )$$

(2) Poon, C. C. Y., and Y. T. Zhang. Cuff-less and noninvasive measurements of arterial blood pressure by pulse transit time. The 2005 IEEE Engineering in Medicine and Biology 27th Annual Conference, Shanghai, China, 2005.

In this paper, equation (1) describes the mathematical relationship between diastolic blood pressure (DBP)/systolic blood pressure (SBP) and pulse wave transit time (PTT)

$$DBP = \frac{\text{?}}{3} + \frac{2 \text{?}}{3} + A ln ( \frac{\text{?}}{{PTT}_{W}} ) - \frac{( \text{?} - \text{?} )}{3} \frac{\text{?}}{{PTT}_{W}^{2}} . \\ SBP = DBP + ( \text{?} - \text{?} ) \frac{\text{?}}{{PTT}_{W}^{2}} \\ \text{?} \text{indicates text missing or illegible when filed} \qquad ( 1 )$$

(3) Muehlsteff, J., X. L. Aubert, and M. Schuett. Cuffless estimation of systolic blood pressure for short effort bicycle tests: the prominent role of the pre-ejection period. The 28th IEEE EMBS Annual International Conference, New York, 2006.

In this paper, equations (3) and (4) describe two different mathematical models between blood pressure (BP) and pulse wave transit time (PTT)

P=A ln PTT+B (3)

P=A(1/PTT)²+B (4)
(4) Yan Chen, Changyun Wen, Guocai Tao, Min Bi, Continuous and noninvasive measurement of systolic and diastolic blood pressure by one mathematical model with the same model parameters and two separate pulse wave velocities, Annals of Biomedical Engineering, 2012, 40(4): 871-882.

In this paper, equation (8) describes how to calculate the pulse wave transit velocity (PWV) through the pulse wave transit time (PTT)

$${PWV}_{s} = \frac{\text{?}}{\text{?}} \\ \text{?} \text{indicates text missing or illegible when filed} \qquad ( 8 )$$

And equation (9) describes how to calculate the SBP through the pulse wave transit velocity (PWVs) related to the systolic pressure.

$$SBP = \text{?} \\ \text{?} \text{indicates text missing or illegible when filed} \qquad ( 9 )$$

And equation (10) describes how to calculate DBP from the pulse wave transit velocity (PWVd) related to diastolic blood pressure.

$$DBP = \text{?} \\ \text{?} \text{indicates text missing or illegible when filed} \qquad ( 10 )$$

(5) Mukkamala R, Hahn J O, Inan O T, Mestha L K, Kim C S, Töreyin H, KyalS. Toward Ubiquitous BloodPressure Monitoring via Pulse Transit Time: Theory and Practice, IEEE Trans Biomed Eng, 2015

In this paper, the mathematical model between blood pressure (BP) and (PTT) is described in equation (12).

$$BP = \frac{{K}_{1}}{{( PTT - {K}_{2} )}^{2}} + {K}_{3} \qquad ( 12 )$$

(6) Xiao-Rong Ding, Yuan-Ting Zhang, Jing Liu, Wen-Xuan Dai, Hon Ki Tsang. Continuous Cuffless Blood Pressure Estimation Using Pulse Transit Time an Photoplethysmogram Intensity Ratio, IEEE Transactions on Biomedical Engineering, 2016 May; 63(5):964-972

In this article, equation (12) describes the mathematical model between systolic blood pressure (SBP) and PTT.

$$SBP = \text{?} \frac{\text{?}}{PIR} + {PP}_{s} \cdot {( \frac{\text{?}}{PTT} )}^{2} \\ \text{?} \text{indicates text missing or illegible when filed} \qquad ( 12 )$$

In a first aspect, a method for correcting PTT associated with diastolic blood pressure, includes the following steps:

S1) detecting a pulse wave at an ear in each cardiac cycle in real time and obtaining the following data: the height of an aortic valve closure point on the ear pulse wave denoted as h_{sd}, the systolic time of the ear pulse wave denoted as tₛ, the diastolic time of the ear pulse wave denoted as t_{d}, and the maximum height of the ear pulse wave denoted as hₘₐₓ;

S2) detecting the pulse wave at a toe in each cardiac cycle in real time and obtaining the following data: the systolic time of the toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between the starting point of the toe pulse wave and the midpoint of the systolic peak of the toe pulse wave denoted as t_{ch-toe}, the time interval between the starting point of the toe pulse wave and the highest point of the systolic peak of the toe pulse wave denoted as tₘₐₓ₋ₜₒₑ, where the midpoint of the peak refers to the midpoint of arising edge turning point and a falling edge turning point at the peak;

h_{sd} refers to the amplitude of the aortic valve closure point of the ear pulse wave relative to the starting point in a cardiac cycle;

hₘₐₓ refers to the maximum amplitude of the systolic peak of the ear pulse wave in a cardiac cycle;

hₘₐₓ₋ₜₒₑ refers to the maximum amplitude of the systolic peak of the toe pulse wave in a cardiac cycle;

tₛ (the systolic time of the ear pulse wave) refers to the time difference between the starting point and the aortic valve closure point on the ear pulse wave in a cardiac cycle;

t_{d} (the diastolic time of the ear pulse wave) refers to the time difference between the aortic valve closure point of the ear pulse wave in one cardiac cycle and the starting point of the ear pulse wave in the next cardiac cycle;

tₛ₋ₜₒₑ (the systolic time of the toe pulse wave) refers to the time difference between the starting point and the aortic valve closure point on the toe pulse wave in a cardiac cycle;

t_{d-toe} (the diastolic time of the toe pulse wave) refers to the time difference between the aortic valve closure point of the toe pulse wave in one cardiac cycle and the starting point of the toe pulse wave in the next cardiac cycle;

S3) calculating the PTT associated with diastolic blood pressure denoted as T_{d}, where T_{d} refers to a time difference between the starting point of the ear pulse wave and the starting point of the toe pulse wave, and h is the amplitude of the ear pulse wave or the toe pulse wave in a longitudinal direction, As for the pulse wave in plane coordinates, the ordinate is amplitude h, the abscissa is time t, and the pulse wave starting point is the coordinate origin, h refers to the height of a specific point on the pulse wave of the ear or toe, not just one of the toe pulse wave or ear pulse wave; h is an unknown quantity, which means the amplitude of the pulse wave at any moment;

S4) by using the data in the same cardiac cycle acquired through step S1 and step S2, calculating a few correction variables b₁-b₇ in the cardiac cycle;

S5) according to the correction variables in the cardiac cycle acquired in step S4, calculating a total correction value in the cardiac cycle; and

S6) continuously acquiring the correction values in a plurality of cardiac cycles, and correcting the I'd acquired in the step S3.

Preferably, the total correction value in the step S5 is

$$B = {\sum }_{{i = 1}^{}}^{7} {b}_{i} ;$$

where B is the sum of the correction variables b₁-b₇, bᵢ is the i-th correction variable.

Preferably, in the step S6, the correction values in 8 cardiac cycles are continuously acquired; a correction method is: T_{dmb}=T_{dm}(1-Bm), where

$${B}_{m} = \frac{1}{8} {\sum }_{{i = 1}^{}}^{8} {B}_{i} , \\ {T}_{d m} = \frac{1}{8} {\sum }_{{i = 1}^{}}^{8} {T}_{di} ,$$

in which T_{dmb} is the T_{d} after correction, T_{dm} is the averaged T_{d} in 8 cardiac cycles, Bₘ is the averaged B in 8 cardiac cycles, Bᵢ is the total correction value in the i-th cardiac cycle, and T_{di} is T_{d} in the i-th cardiac cycle.

Preferably, the first correction variable b₁ is calculated by the following formulas:
if d_{1-b}≤k_{sd-m-0}≤d_{1-2-b}, then b₁=(d_{1-2-b}−k_{sd-m-0})×0.4;
if k_{sd-m-0}<d_{1-b}, then b₁=24×0.4;
if k_{sd-m-0}>d_{1-2-b}, then b₁=0;
where d_{1-b}=74 to 82, d_{1-2,b}=98 to 106,

$${k}_{sd - m - 0} = \frac{{t}_{s} {h}_{sd}}{{\int }_{0}^{{t}_{s}} hdt} .$$

Preferably, the second correction variable b₂ is calculated by the following formulas:
if k_{sd-m}>(d_{2-b}+(age-14)/15/100), then b₂=(k_{sd-m}−(d_{2-b}+(age-14)/15/100))×0.5;
if k_{sd-m}≤(d_{2-b}+(age-14)/15/100), then b₂=0;
where d_{2-b}=1.33 to 1.43, age is age; if |k_{sd-m-0}−k_{sd-m-ts}|≥40 and (k_{sd-m-0}+k_{sd-m-ts})/2≥k_{sd-m-2}, then k_{sd-m}=2×k_{sd-m-2}-(k_{sd-m-0}+k_{sd-m-ts})/2, otherwise k_{sd-m}=k_{sd-m-2};

$${k}_{sd - m - 0} = \frac{{t}_{s} {h}_{sd}}{{\int }_{0}^{{t}_{s}} hdt} , {k}_{sd - m - ts} = \frac{{t}_{s} {h}_{sd}}{{\int }_{{t}_{s}}^{2 {t}_{s}} hdt} , {k}_{sd - m - 2} = \frac{2 {t}_{s} {h}_{sd}}{{\int }_{0}^{2 {t}_{s}} hdt} .$$

Preferably, the third correction variable b₃ is calculated by the following formulas:
if c₄<k_{d-m-a}<c₅, then b₃=0;
if k_{sd-m-0}<d₆ or k_{sd-m-2}>d₇, then b₃=0;
if k_{sd-m-0}≥d₆+0.10 and k_{sd-m-2}≤d₈ and k_{d-m-a}≤c₄, then b₃=(c₄−k_{d-m-a})×67/100;
if

$$\{ \begin{matrix}{d}_{6} \leq {k}_{sd - m - 0} < {d}_{6} + 0.1 \\ {k}_{d - m - a} \leq {c}_{4}\end{matrix} or$$

$$\{ \begin{matrix}{d}_{8} < {k}_{sd - m - 2} < {d}_{7} \\ {k}_{d - m - a} \leq {c}_{4}\end{matrix} ,$$

then b₃=(c₄−k_{d-m-a})×50/100;
if k_{sd-m-0}≥d₆+0.10 and k_{sd-m-2}≤d₈ and k_{d-m-a}≥c₅, then b₃=(c₅−k_{d-m-a})×62/100;
if

$$\{ \begin{matrix}{d}_{6} \leq {k}_{sd - m - 0} < {d}_{6} + 0.1 \\ {k}_{d - m - a} \geq {c}_{5}\end{matrix} or$$

$$\{ \begin{matrix}{d}_{8} < {k}_{sd - m - 2} < {d}_{7} \\ {k}_{d - m - a} \geq {c}_{5}\end{matrix} ,$$

then b₃=(c₅−k_{d-m-a})×45/100;
where if |k_{sd-m-0}−k_{sd-m-ts}|≥40 and (k_{sd-m-0}+k_{sd-m-ts})/2≥k_{sd-m-2} and k_{sd-m-ts}≥d₃₋₂, then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2, otherwise k_{d-m-a}=(k_{d-m-t}_{_{d}}₋₁+k_{d-m-t}_{_{d-toe}})/2
if k_{sd-m-ts}≤d₃₋₂, then k_{d-m-t}_{_{d}}₋₁=k_{d-m-t}_{_{d}}(d₃₋₂−k_{sd-m-ts})×75/100; if k_{d-m-t}_{_{d}}≤d₃, then k_{d-m-t}_{_{d-1}}=d₃; if k_{d-m-t}_{_{d-toe}}≤d₃, then k_{d-m-t}_{_{d-toe}}=d₃;

$${k}_{d - m - {t}_{d}} = \frac{{\int }_{{t}_{s}}^{{t}_{s} + {t}_{d}} hdt}{{t}_{d} {h}_{max}} , {k}_{sd - m - 0} = \frac{{t}_{s} {h}_{sd}}{{\int }_{0}^{{t}_{s}} hdt} , {k}_{sd - m - 2} = \frac{2 {t}_{s} {h}_{sd}}{{\int }_{0}^{2 {t}_{s}} hdt} ,$$

$${k}_{sd - m - ts} = \frac{{t}_{s} {h}_{sd}}{{\int }_{{t}_{s}}^{2 {t}_{s}} hdt} ,$$

c₄=(d₄+(age-14)/8)/100, d₄=23 to 35, c₅=(d₅+(age-14)/8)/100, d₅=27 to 39, d₆=0.97 to 1.03, d₇=1.52 to 1.58, d₈=1.42 to 1.48, d₃₋₂=1.21 to 1.31, d₃=0.02 to 0.14, and age is age.

Preferably, the fourth correction variable b₄ is calculated by the following formulas:
if kₛ₋ₜ₋ₜₒₑ>0.8, then b₄=kₛ₋ₜ₋ₜₒₑ−0.8;
if kₛ₋ₜ₋ₜₒₑ≤0.8, then b₄=0;

where if tₘₐₓ₋ₜₒₑ≥t_{ch-toe}, then

$${k}_{s - t - toe} = \frac{{t}_{max - toe} + {t}_{ch - toe} + 400}{( {t}_{s - toe} + 200 ) \times 2} ,$$

otherwise

$${k}_{s - t - toe} = \frac{{t}_{max - toe} + 200}{{t}_{s - toe} + 200} .$$

Preferably, the fifth correction variable b₅ is calculated by the following formulas:

if kₛ₋ₘ₋ₜₒₑ<d₉, then b₅=0;

if kₛ₋ₘ₋ₜₒₑ≥d₉ and kₛ₋ₜ₋ₜₒₑ≥0.8, then b₅=kₛ₋ₘ₋ₜₒₑ−d₉;

if kₛ₋ₘ₋ₜₒₑ≥d₉ and kₛ₋ₜ₋ₜₒₑ<0.8, then b₅=(kₛ₋ₘ₋ₜₒₑ−d₉)/2;

where d₉=0.67 to 0.73,

$${k}_{s - m - toe} = \frac{{\int }_{0}^{{t}_{s - toe}} h dt}{{t}_{s - toe} {h}_{max - toe}} .$$

Preferably, the sixth correction variable b₆ is calculated by the following formulas:

if kₛ₋ₘ₋ₜₒₑ₋ₑₐᵣ<1.0, then b₆=0;

when kₛ₋ₘ₋ₜₒₑ₋ₑₐᵣ>1.08, then c₆=1.08, meantime, if tₛ>220 and k_{sd-m-0}>0.88, then b₆=c₆−1.0, if tₛ<160 or k_{sd-m-0}<0.80, then b₆=(c₆−1.0)×0.34, if 160<tₛ≤220 or 0.80<k_{sd-m-0}≤0.88, then b₆=(c₆−1.0)×0.67;

when 1.0≤kₛ₋ₘ₋ₜₒₑ₋ₑₐᵣ≤1.08, then c₆=kₛ₋ₘ₋ₜₒₑ₋ₑₐᵣ−1.0, meantime, if tₛ>220 and k_{sd-m-0}>0.88, then b₆=c₆, if tₛ≤160 or k_{sd-m-0}≤0.80, then b₆=c₆×0.34, if 160<tₛ≤220 or 0.80<k_{sd-m-0}≤0.88, then b₆=c₆×0.67;

$${k}_{s - m - toe - ear} = \frac{{h}_{max} {\int }_{0}^{{t}_{s - toe}} h dt ( {t}_{s} - {t}_{s - toe} ) \times 100}{{h}_{max - toe} {\int }_{0}^{{t}_{s}} h dt ( {t}_{s} - {t}_{s - toe} ) \times 100} ,$$

$${k}_{s - m - 0} = \frac{{t}_{s} {h}_{sd}}{{\int }_{{t}_{s}}^{{t}_{s}} hdt} .$$

where

Preferably, the seventh correction variable b₇ is calculated by the following formulas:
if kₜₛ₋ₜₒₑ₋ₑₐᵣ<1.0, then b₇=0;
when kₜₛ₋ₜₒₑ₋ₑₐᵣ>1.08, then c₇=1 0.08, meantime, if tₛ>220 and k_{sd-m-0}>0.88, then b₇=c₇−1.0, if tₛ<160 or k_{sd-m-0}<0.80, then b₇=(c₇−1.0)×0.34, if 160<tₛ≤220 or 0.80<k_{sd-m-0}≤0.88, then b₇=(c₇−1.0)×0.67;
when 1.0≤kₜₛ₋ₜₒₑ₋ₑₐᵣ≤1.08, then c₇=kₜₛ₋ₜₒₑ₋ₑₐᵣ−1.0, meantime, if tₛ>220 and k_{sd-m-0}>0.88, then b₇=c₇, if tₛ≤160 or k_{sd-m-0}≤0.80, then b₇=c₇×0.34, if 160<tₛ≤220 or 0.80<k_{sd-m-0}≤0.88, then b₇=c₇×0.67;
where

$${k}_{ts - toe - ear} = \frac{{t}_{s - toe} + 825}{{t}_{s} + 825} , {k}_{sd - m - 0} = \frac{{t}_{s} {h}_{sd}}{{\int }_{0}^{{t}_{s}} hdt} .$$

What described above is the method for correcting PTT associated with diastolic blood pressure. The PTT associated with diastolic blood pressure is determined by a time difference of an ear pulse wave and a toe pulse wave in the same cardiac cycle, and a few correction variables are extracted based on the pulse wave features, then a total correction value is acquired to perform adaptive correction on the irregular change of pulse wave transit time. The corrected transit time can be used with available mathematical models for continuously and accurately measuring diastolic blood pressure in each cardiac cycle in a clinical setting.

In a second aspect, a method for correcting PTT associated with systolic blood pressure includes the following steps:

S1) detecting a pulse wave at an ear in each cardiac cycle in real time and obtaining the following data: the height of an aortic valve closure point on the ear pulse wave denoted as h_{sd}, the systolic time of the ear pulse wave denoted as tₛ, the diastolic time of the ear pulse wave denoted as t_{d}, and the maximum height of the ear pulse wave denoted as hₘₐₓ;

h_{sd} refers to the amplitude of the aortic valve closure point of the ear pulse wave relative to the starting point in a cardiac cycle;

hₘₐₓ refers to the maximum amplitude of the systolic peak of the ear pulse wave in a cardiac cycle;

tₛ (the systolic time of the ear pulse wave) refers to the time difference between the starting point and the aortic valve closure point on the ear pulse wave in a cardiac cycle;

t_{d} (the diastolic time of the ear pulse wave)refers to the time difference between the aortic valve closure point of the ear pulse wave in one cardiac cycle and the starting point of the ear pulse wave in the next cardiac cycle;

S2) detecting the pulse wave at a toe in each cardiac cycle in real time and obtaining the following data: the systolic time of the toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between the starting point to the midpoint of the peak of the toe pulse wave denoted as t_{ch-toe}, the time interval between the starting point to the highest point of the peak of the toe pulse wave denoted as tₘₐₓ₋ₜₒₑ, where the midpoint of the peak refers to the midpoint of arising edge turning point and a falling edge turning point at the peak;

hₘₐₓ₋ₜₒₑ refers to the maximum amplitude of the systolic peak of the toe pulse wave in a cardiac cycle;

tₛ₋ₜₒₑ (the systolic time of the toe pulse wave) refers to the time difference between the starting point and the aortic valve closure point on the toe pulse wave in a cardiac cycle;
t_{d-toe} (the diastolic time of the toe pulse wave) refers to the time difference between the aortic valve closure point of the toe pulse wave in one cardiac cycle and the starting point of the toe pulse wave in the next cardiac cycle;

S3) calculating the PTT associated with systolic blood pressure denoted as Tₛ, where Tₛ refers to a time difference between the aortic valve closure point on the ear pulse wave and the aortic valve closure point on the toe pulse wave, and h is the amplitude of the ear pulse wave or the toe pulse wave in a longitudinal direction;

S4) by using the data in the same cardiac cycle acquired through step S1 and step S2, calculating a few correction variables a₁-a₇ in the cardiac cycle;

S5) according to the correction variables in the cardiac cycle acquired in step S4, calculating a total correction value in the cardiac cycle; and

S6) continuously acquiring the correction values in a plurality of cardiac cycles, and correcting the Tₛ acquired in the step S3.

Preferably, the total correction value in the step S5 is

$$A = {\sum }_{i = 1}^{7} {a}_{i} ,$$

where A is the sum of the correction variables a₁-a₇, aᵢ is the i-th correction variable.

Preferably, in the step S6, the correction values in 8 cardiac cycles are continuously acquired; a correction method is: Tₛₘₐ=Tₛₘ(1−Aₘ); where

$${A}_{m} = \frac{1}{8} {\sum }_{i = 1}^{8} {A}_{i} , {T}_{sm} = \frac{1}{8} {\sum }_{i = 1}^{8} {T}_{si} ,$$

in which Tₛₘₐ is the Tₛ after correction, Tₛₘ is the averaged Tₛ in 8 cardiac cycles, Aₘ is the averaged A in 8 cardiac cycles, Aᵢ is the total correction value in the i-th cardiac cycle, and Tₛᵢ is Tₛ in the i-th cardiac cycle.

Preferably, the first correction variable a₁ is calculated by the following formulas:
if d₁≤k_{sd-m-0}≤d₁₋₂, then a₁=(d₁₋₂−k_{sd-m-0})×0.50;
if k_{sd-m-0}<d₁, then a₁=28×0.50;
if k_{sd-m-0}>d₁₋₂, then a₁=0;
where

$${k}_{s - m - 0} = \frac{{t}_{s} {h}_{sd}}{{\int }_{0}^{{t}_{s}} hdt} ,$$

d₁=76 to 84, and d₁₋₂₌₁₀₄ to 112.

Preferably, the second correction variable az is calculated by the following formulas:
if k_{sd-m}>(d₂+(age-14)/15/100), then a₂=k_{sd-m}−(d₂+(age-14)/15/100);
if k_{sd-m}≤(d₂+(age-14)/15/100), then a₂=0;
where if |k_{sd-m-0}−k_{sd-m-ts}|≥40 and (k_{sd-m-0}+k_{sd-m-ts})/2≥k_{sd-m-2}, then k_{sd-m}=2×k_{sd-m-2}−(k_{sd-m-0}+k_{sd-m-ts})/2, otherwise k_{sd-m}=k_{sd-m-2};

$${k}_{s - m - 0} = \frac{{t}_{s} {h}_{sd}}{{\int }_{0}^{{t}_{s}} hdt} , {k}_{sd - m - ts} = \frac{{t}_{s} {h}_{sd}}{{\int }_{{t}_{s}}^{2 {t}_{s}} hdt} , {k}_{sd - m - 2} = \frac{2 {t}_{s} {h}_{sd}}{{\int }_{0}^{2 {t}_{s}} hdt} ,$$

age is age, and d₂=1.17 to 1.27.

Preferably, the third correction variable a₃ is calculated by the following formulas:
if c₄<k_{d-m-a}<c₅, then a₃=0;
if k_{sd-m-0}<d₆ or k_{sd-m-2}>d₇, then a₃=0;
if k_{sd-m-0}≥d₆+0.10 and k_{sd-m-2}≤d₈ and k_{d-m-a}≤c₄, then a₃=(c₄−k_{d-m-a})×67/100;
if

$$\{ \begin{matrix}{d}_{6} \leq {k}_{sd - m - 0} < {d}_{6} + 0.1 \\ {k}_{d - m - a} \leq {c}_{4}\end{matrix}$$

$$\{ \begin{matrix}{d}_{8} < {k}_{sd - m - 2} < {d}_{7} \\ {k}_{d - m - a} \leq {c}_{4}\end{matrix} ,$$

then a₃=(c₄−k_{d-m-a})×50/100;
if k_{sd-m-0}≥d₆+0.10 and k_{sd-m-2}≤d₈ and k_{d-m-a}≥c₅, then a₃=(c₅−k_{d-m-a})×62/100;
if

$$\{ \begin{matrix}{d}_{6} \leq {k}_{sd - m - 0} < {d}_{6} + 0.1 \\ {k}_{d - m - a} \geq {c}_{5}\end{matrix} or \{ \begin{matrix}{d}_{8} < {k}_{sd - m - 2} < {d}_{7} \\ {k}_{d - m - a} \geq {c}_{5}\end{matrix} ,$$

then a₃=(c₅−k_{d-m-a})×45/100;
where if |k_{sd-m-0}−k_{sd-m-ts}|≥40 and (k_{sd-m-0}+k_{sd-m-ts})/2≥k_{sd-m-2} and k_{sd-m-ts}≥d₃₋₂, then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2, otherwise k_{d-m-a}=(k_{d-m-t}_{_{d}}₋₁+k_{d-m-t}_{_{d-toe}})/2;
if k_{sd-m-ts}≤d₃₋₂, then k_{d-m-t}_{_{d}}₋₁=k_{d-m-t}_{_{d}}−(d₃₋₂−k_{sd-m-ts})×75/100; if k_{d-m-t}_{_{d}}≤d₃, then k_{d-m-t}_{_{d}}₋₁=d₃; if k_{d-m-t}_{_{d-toe}}≤d₃, then k_{d-m-t}_{_{d-toe}}=d₃;

$${k}_{d - m - {t}_{d}} = \frac{{\int }_{{t}_{s}}^{{t}_{s} + {t}_{d}} h d t}{{t}_{d} {h}_{max}} , {k}_{s d - m - 0} = \frac{{t}_{s} {h}_{s d}}{{\int }_{0}^{{t}_{s}} h d t} , {k}_{s d - m - 2} = \frac{2 {t}_{s} {h}_{s d}}{{\int }_{0}^{2 {t}_{s}} h d t} , {k}_{sd - m - ts} = \frac{{t}_{s} {h}_{s d}}{{\int }_{{t}_{s}}^{2 {t}_{s}} h d t} , {k}_{d - m - {t}_{d - toe}} = \frac{{\int }_{{t}_{s - toe}}^{{t}_{s - toe} + {t}_{d - toe}} h d t}{{t}_{d - t o e} {h}_{max - toe}}$$

c₄=(d₄+(age-14)/8)/100, d₄=23 to 35, c₅=(d₅+(age-14)18)/100, d₅=27 to 39, d₆=0.97 to 1.03, d₇=1.52 to 1.58, d₈=1.42 to 1.48, d₃₋₂=1.21 to 1.31, d₃=0.02 to 0.14, and age is age.

Preferably, the fourth correction variable a₄ is calculated by the following formulas: if kₛ₋ₜ₋ₜₒₑ>0.8, then as=kₛ₋ₜ₋ₜₒₑ-0.8;
if kₛ₋ₜ₋ₜₒₑ≤0.8, then a₄=0;
where if tₘₐₓ₋ₜₒₑ≥t_{ch-toe}, then

$${k}_{s - t - t o e} = \frac{{t}_{max - toe} + {t}_{c h - t o e} + 400}{( {t}_{s - t o e} + 2 \; 00 ) ⨯ 2} ,$$

otherwise

$${k}_{s - t - t o e} = \frac{{t}_{max - toe} + 200}{{t}_{s - t o e} + 200} .$$

Preferably, the fifth correction variable as is calculated by the following formulas:
if kₛ₋ₘ₋ₜₒₑ<d₉, then a₅=0;
if kₛ₋ₘ₋ₜₒₑ≥d₉ and kₛ₋ₜ₋ₜₒₑ≥0.8, then a₅=kₛ₋ₘ₋ₜₒₑ−d₉;
if kₛ₋ₘ₋ₜₒₑ≥d₉ and kₛ₋ₜ₋ₜₒₑ<0.8, then a₅=(kₛ₋ₘ₋ₜₒₑ−d₉)/2;
where d₉=0.67 to 0.73,

$${k}_{s - m - toe} = \frac{{\int }_{0}^{{t}_{s - toe}} hdt}{{t}_{s - toe} {h}_{max - toe}} .$$

Preferably, the sixth correction variable a₆ is calculated by the following formulas:
if kₛ₋ₘ₋ₜₒₑ₋ₑₐᵣ<1.0, then a₆=0;
when kₛ₋ₘ₋ₜₒₑ₋ₑₐᵣ>1.08, then c₆=1.08, meantime, if tₛ>220 and k_{sd-m-0}>0.88, then a₆=c₆−1.0, if tₛ<160 or k_{sd-m-0}<0.80, then a₆=(c₆−1.0)×0.34, if 160<tₛ≤220 or 0.80<k_{sd-m-0}≤0.88, then a₆=(c₆−1.0)×0.67;
when 1.0<kₛ₋ₘ₋ₜₒₑ₋ₑₐᵣ≤1.08, then c₆=kₛ₋ₘ₋ₜₒₑ₋ₑₐᵣ−1.0, meantime, if tₛ>220 and k_{sd-m-0}>0.88, then a₆=c₆, if tₛ<160 or k_{sd-m-0}≤0.80, then a₆=c6×0.34, if 160<tₛ≤220 or 0.80<k_{sd-m-0}≤0.88, then a₆=c₆×0.67;
where

$${k}_{s - m - toe - ear} = \frac{{h}_{max} {\int }_{0}^{{t}_{s - toe}} hdt + ( {t}_{s} + {t}_{s - t o e} ) ⨯ 100}{{h}_{max - toe} {\int }_{0}^{{t}_{s}} hdt + ( {t}_{s} + {t}_{s - t o e} ) ⨯ 100} , {k}_{s d - m - 0} = \frac{{t}_{s} {h}_{s d}}{{\int }_{0}^{{t}_{s}} hdt} .$$

Preferably, the seventh correction variable a₇ is calculated by the following formulas:
if kₜₛ₋ₜₒₑ₋ₑₐᵣ<1.0, then a₇=0;
when kₜₛ₋ₜₒₑ₋ₑₐᵣ>1.08, then c₇=1.08, meantime, if tₛ>220 and k_{sd-m-0}>0.88, then a₇=c₇−1.0, if tₛ<160 or k_{sd-m-0}<0.80, then a₇=(c₇−1.0)×0.34, if 160<tₛ≤220 or 0.80<k_{sd-m-0}≤0.88, then a₇=(c₇−1.0)×0.67;
when 1.0≤kₜₛ₋ₜₒₑ₋ₑₐᵣ≤1.08, then c₇=kₜₛ₋ₜₒₑ₋ₑₐᵣ−1.0, meantime, if tₛ>220 and k_{sd-m-0}>0.88, then a₇=c₇, if tₛ≤160 or k_{sd-m-0}≤0.80, then a₇=c₇×0.34, if 160<tₛ≤220 or 0.80<k_{sd-m-0}≤0.88, then a₇=c₇×0.67;

where

$${k}_{t s - t o e - e a r} = \frac{{t}_{s - t o e} + 825}{{t}_{s} + 825} , {k}_{s d - m - 0} = \frac{{t}_{s} {h}_{s d}}{{\int }_{0}^{{t}_{s}} hdt} .$$

What described above is the method for correcting PTT associated with systolic blood pressure. The PTT associated with systolic blood pressure is determined by a time difference of an ear pulse wave and a toe pulse wave in the same cardiac cycle, and a few correction variables are extracted based on the pulse wave features, then a total correction value is acquired to perform adaptive correction on the irregular change of pulse wave transit time. The corrected transit time can be used with available mathematical models for continuously and accurately measuring systolic blood pressure in each cardiac cycle in a clinical setting.

The present invention provides a method for correcting Td and Ts. By using the 7 correction variables, it is possible to self-adjust the changes in PTT caused by blood transfusion, vasoactive drugs, surgical intervention and other factor sunder clinical conditions. The corrected Td and Ts can be used in existing mathematical models (either the author's own mathematical model, or the mathematical model proposed by other studies), and continuously measure the diastolic and systolic blood pressure in each cardiac cycle under clinical conditions (especially surgical conditions), with high accuracy.

### DESCRIPTION OF THE EMBODIMENTS

The embodiments of the technical solution of the present invention will be described in detail below. The following embodiments are only used for more clearly illustrating the technical solutions of the present invention, and thus are only examples, and not intended to limit the protection scope of the present invention.

Pulse wave transit time (PTT) changes in perioperative period can be divided into two categories: type I changes: PTT changes caused by changes in blood pressure; and type II changes: unsynchronized changes in PTT and blood pressure (the direction or amount of changes of the two does not conform to a regular function rule). For example, when the blood volume is mildly insufficient, PTT will increase, but due to the adjustment on peripheral resistance of the body, blood pressure may not change much. The use of a hook in thoracoabdominal surgery may seriously affect PTT, but has less effect on blood pressure. Norepinephrine makes small arteries strongly contract and the blood pressure significantly increase, but the effect on the average PTT of the whole body is small.

When PTT has type I changes, the relationship between PTT and blood pressure can still be expressed by a certain function, and the change in blood pressure can be estimated by a mathematical model. While when PTT has type II changes, using a mathematical model based on a conventional circulatory system to estimate blood pressure will produce large errors. The errors are principle errors in measurement of blood pressure by using PTT and cannot be solved by initial calibration and periodic calibration of mathematical model parameters. The difference in PTT among different individuals and the irregular change of PTT in the same individual are two different types of problems, which need to be solved by different methods. To this end, the present invention extracts various variables based on the features of the pulse wave to identify and adaptively correct various type II changes of the PTT, and overcome the principle errors; the available mathematical models can be combined to form a continuous and non-invasive measurement method of blood pressure with an adaptive calibration function, without the need to rely on conventional methods such as oscillometry for repeated calibration.

Positions of the human body for detecting the pulse wave are preferably the ear and the toe. The pulse waves of these two parts contain the physiological and pathological information of the aorta and peripheral arteries, and are representative in propagation paths. A sensor for detecting the pulse signal is preferably an infrared photo plethysmograph (PPG).

The feature changes of ear and toe pulse waves, and the relative changes in features between the two pulse waves provide rich information for identifying the type II changes in PTT and the changes in blood pressure difference between different sites of the body. The present invention collects the invasive arterial blood pressure, pulse waves of the ears and toes, and PTT of a large number of surgical cases for several years for analysing, extracts various variables according to the feature changes and relative feature changes of the two pulse waves, studies the relationship between different variables and different type II changes of PTT, and defines the application scopes of these variables.

In clinical application, during continuous measurement of blood pressure using PPT, the pulse waveform is analysed in real time and variables are extracted. Whether the PTT has the type II changes is determined according to whether the variables fall within the application scope, and the nature and extent of the type II changes of the PTT are determined according to the nature of the applicable variables. If a variable is outside the application scope, the corresponding type II changes do not occur in the PTT, then the variable is not applicable. Several applicable variables are fused to calculate the correction amount to correct the PTT. The corrected PTT/PWV is applicable to the available mathematical models to accurately calculate the blood pressure.

The present invention uses limited variables to express the most important changes of pulse wave form, and studies the relationship between these changes and PTT. As for the pulse wave in plane coordinates, the ordinate is amplitude h, the abscissa is time t, and the pulse wave starting point is the coordinate origin.

### Embodiment 1

A method for correcting PTT associated with diastolic blood pressure, includes the following steps:

S1) detecting a pulse wave at an ear in each cardiac cycle in real time and obtaining the following data: the height of an aortic valve closure point on an ear pulse wave denoted as h_{sd}, that is, the height at a junction between the systolic and diastolic phases on the ear pulse wave, the systolic time of the ear pulse wave denoted as tₛ, the diastolic time of the ear pulse wave denoted as t_{d}, and the maximum height of the ear pulse wave denoted as hₘₐₓ;

S2) detecting the pulse wave at a toe in each cardiac cycle in real time and obtaining the following data: the systolic time of the toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between the starting point to the midpoint of the peak of the toe pulse wave denoted as t_{d-toe}, and the time interval between the starting point to the peak of the toe pulse wave denoted as tₘₐₓ₋ₜₒₑ, where the midpoint of the peak refers to the midpoint of arising edge turning point and a falling edge turning point at the peak; the definition of the midpoint of the peak can be understood by referring to the literature YAN CHEN, CHANGYUN WEN, GUOCAI TAO, and MIN BI Continuous and Non-invasive Measurement of Systolic and Diastolic Blood Pressure by One Mathematical Model with the Same Model Parameters and Two Separate Pulse Wave Velocities.

S3) calculating the PTT associated with diastolic blood pressure denoted as T_{d}, and the definition can be understood by referring to the literature YAN CHEN, CHANGYUN WEN, GUOCAI TAO, and MIN BI Continuous and Non-invasive Measurement of Systolic and Diastolic Blood Pressure by One Mathematical Model with the Same Model Parameters and Two Separate Pulse Wave Velocities; h is the amplitude of the ear pulse wave or the toe pulse wave in a longitudinal direction;

S4) by using the data in the same cardiac cycle acquired through step S1 and step S2, calculating a few correction variables in the cardiac cycle;

S5) according to the correction variables in the cardiac cycle acquired in step S4, calculating a total correction value in the cardiac cycle; and

S6) continuously acquiring the correction values in a plurality of cardiac cycles, and correcting the T_{d} acquired in the step S3.

By the method, the PTT associated with diastolic blood pressure is determined by a time difference of an ear pulse wave and a toe pulse wave in the same cardiac cycle, and a few correction variables are extracted based on the pulse wave features, then a total correction value is acquired to perform adaptive correction on the irregular change of pulse wave transit time. The corrected transit time can be used with available mathematical models for continuously and accurately measuring diastolic blood pressure in each cardiac cycle in a clinical setting.

First correction variable b₁:

The correction variables obtained in the step S4 include a first correction variable b₁. b₁ is used for correcting the type II changes in T_{d} in a hypotensive state, the applicable range of b₁ is b₁>0, and if b₁ is larger, the blood pressure is lower.

$${k}_{s d - m - 0} = \frac{{t}_{s} {h}_{s d}}{{\int }_{0}^{{t}_{s}} h d t} ,$$

k_{sd-m-0} represents the ratio of h_{sd} to the average height of the ear pulse wave systole. In some cases, under a hypotensive state, the pulse wave peak appears as a forward-inclined triangle; When h_{sd} decreases a lot, k_{sd-m-0} becomes smaller, indicating that the waveform at the end of the aortic systole is much lower, the continuous power for pushing pulse wave transit is insufficient, and the transit time is prolonged. In this state, the diastolic information is unstable and should not be used.

d_{1-b}=74 to 82, preferably is 78. d_{1-2-b}=98 to 106, preferably is 102.

When the continuous power for pushing pulse wave transit is insufficient, the transit time T_{d} is prolonged and needed be corrected by b₁. That is, if d_{1-b}≤k_{sd-m-0}≤d_{1-2-b}, then b₁=(d_{1-2-b}−k_{sd-m-0})×0.4.

When the continuous power for pushing pulse wave transit is seriously insufficient, the transit time T_{d} is prolonged a lot, and b₁ takes the upper limit value for correction. That is, if k_{sd-m-0}<d_{1-b}, then b₁=24×0.4.

When the continuous power for pushing pulse wave transit is sufficient, T_{d} does not need to be corrected, and b₁ is not applicable. That is, if k_{sd-m-0}>d_{1-2-b}, then set b₁=0.

Second correction variable b₂:

The correction variables obtained in the step S4 also include a second correction variable b₂, b₂ is used for correcting the type II changes in T_{d} in a hypertensive state, the applicable range of b₂ is b_{2>0}, and if b₂ is larger, the diastolic blood pressure is higher.

$${k}_{sd - m - ts} = \frac{{t}_{s} {h}_{s d}}{{\int }_{{t}_{s}}^{2 {t}_{s}} h d t} ,$$

k_{sd-m-ts} represents the ratio of h_{sd} to the average height of the tₛ to 2tₛ segments of the ear pulse wave diastole, and is used for determining the irregular change of the pulse wave diastole. For example, in a thoracoabdominal surgery, an upward pulling hook causes the aortic stress to change, so that the amplitude of the ear pulse wave diastole is reduced, and the k_{sd-m-ts} becomes larger.

$${k}_{s d - m - 2} = \frac{2 {t}_{s} {h}_{s d}}{{\int }_{0}^{2 {t}_{s}} hdt} ,$$

k_{sd-m-2} represents the ratio of h_{sd} to the average height of the ear pulse wave 0 to 2tₛ segments, includes the information of systolic and partial diastolic waveform, and is mainly used for a hypertensive state, such as increase of heart rate and blood pressure caused by tracheal intubation. In the state of hypertension, the ear pulse wave appears an equilateral triangle or a backward-inclined triangle, h_{sd} rises a lot, and k_{sd-m-2} becomes larger. Compared with the waveform in a normal blood pressure state, the slope of the rising edge of the waveform in the hypertensive state becomes smaller, the power for pushing pulse wave transit is insufficient, and the transit time T_{d} is prolonged.

If |k_{sd-m-0}−k_{sd-m-ts}|≥40 and (k_{sd-m-0}+k_{sd-m-ts})/2≥k_{sd-m-2},

then k_{sd-m}=2×k_{sd-m-2}−(k_{sd-m-0}+k_{sd-m-ts})/2,

otherwise k_{sd-m}=k_{sd-m-2};

If the waveform of the ear pulse wave diastole has an irregular change, for example, if the upward pulling hook of the thoracoabdominal surgery causes the aortic stress to change, and the form of the pulse wave diastole changes significantly, k_{sd-m} is corrected, otherwise k_{sd-m}=k_{sd-m-2}.

d_{2-b}=1.33 to 1.43, preferably is 1.38.

If k_{sd-m}>(d_{2-b}+(age-14)/15/100), where age is age, the continuous power corresponding to the diastolic pressure is insufficient, the transit time T_{d} is relatively prolonged, and needed be corrected by b₂, then b₂=(k_{sd-m}−(d_{2-b}+(age-14)/15/100))×0.5, the change of b₂ is inversely proportional to the change of the slope of the pulse wave rising edge, where 0.5 is the proportional coefficient.

If k_{sd-m}≤(d_{2-b}+(age-14)/15/100), the continuous power corresponding to the diastolic pressure is sufficient, b₂ is not applicable, then set b₂=0.

Third correction variable b₃:

The correction variables obtained in the step S4 further include a third correction variable b₃, which is used for correcting the T_{d} in a state that the blood volume changes or the body temperature of a sensor placement site changes.

$${k}_{d - m - {t}_{d}} = \frac{{\int }_{{t}_{s}}^{{t}_{s} + {t}_{d}} h d t}{{t}_{d} {h}_{max}} ,$$

k_{d-m-t}_{_{d}} is the ratio of the average height of the ear pulse wave diastole to the maximum height hₘₐₓ. The blood volume is reduced when a patient fasts and drinks less water before surgery, k_{d-m-t}_{_{d}} decreases, and the transit time is prolonged; when the blood volume increases due to blood transfusion and IV transfusion in the operation, k_{d-m-t}_{_{d}} increases, and the transit time is shortened.

If k_{sd-m-ts}≤d₃₋₂, indicating that the early diastole of ear pulse wave rises and exceeds a normal range, then k_{d-m-t}_{_{d}} needs to be corrected, and the correction result is noted as k_{d-m-t}_{_{d}}₋₁.

k_{d-m-t}_{ₜ}₋₁=k_{d-m-t}_{_{d}}−(d₃₋₂−k_{sd-m-ts})×75/100; if k_{d-m-t}_{_{d}}≤d₃, indicated that the ear pulse wave is disturbed, then k_{d-m-t}_{_{d}}₋₁=d₃. d₃=0.02 to 0.14, preferably is 0.08; d₃₋₂=1.21 to 1.31, preferably is 1.26.

$${k}_{d - m - {t}_{d - toe}} = \frac{{\int }_{{t}_{s - toe}}^{{t}_{s - toe} + {t}_{d - toe}} hdt}{{t}_{d - t o e} {h}_{max - toe}} ,$$

k_{d-m-t}_{_{d-toe}} is the ratio of the average height of the toe pulse wave diastole to the maximum height hₘₐₓ₋ₜₒₑ, tₛ₋ₜₒₑ represents the systolic time of the toe pulse wave, and t_{d-toe} represents the diastolic time of the toe pulse wave. If k_{d-m-t}_{_{d-toe}}≤d₃, then k_{d-m-t}_{_{d-toe}}=d₃. The properties of k_{d-m-t}_{_{d-toe}} and k_{d-m-t}_{_{d}} are same.

k_{d-m-a}=(k_{d-m-t}_{_{d}}₋₁+k_{d-m-t}_{_{d-toe}})=/2 two variables from the ear and toe pulse waves with the same property are combined, and the average value is taken as a correction variable; if the pulse wave diastole has an irregular change, the k_{d-m-a} is corrected.

If |k_{sd-m-0}−k_{sd-m-ts}|≥40 and (k_{sd-m-0}+k_{sd-m-ts})/2≥k_{sd-m-2} and k_{sd-m-ts}≥d₃₋₂,

then k_{d-m-a}=(k_{d-m-t}_{_{d}}₋₁+k_{d-m-t}_{_{d-toe}}+(k_{sd-m-0}++k_{sd-m-ts})/2−k_{sd-m-2})/2.

In the state that the blood volume is normal and the body temperature of the sensor placement site is also normal, b₃ is not applicable. That is, if c₄<k_{d-m-a}<c₅, then set b₃=0. c₄=(d₄+(age-14)/8)/100, d₄=23 to 35, preferably is 29; c₅=(d₅+(age-14)/8)/100, d₅=27 to 39, preferably is 33.

In an extremely low or high blood pressure state, the information of diastolic period is unstable, and b₃ is not applicable. That is, if k_{sd-m-0}<d₆ or k_{sd-m-2}>d₇, then set b₃=0. d₆=0.97 to 1.03, preferably is 1.00; d₇=1.52 to 1.58, preferably is 1.55.

In a normal blood pressure state, when the blood volume decreases or the body temperature of the sensor placement site decreases, b₃ takes 67% of a positive value. That is, if k_{sd-m-0}≥d₆+0.10 and k_{sd-m-2}≤d₈ and k_{d-m-a}≤c₄, then b₃=(c₄−k_{(d-m-a)}×67/100. d₈=1.42 to 1.48, preferably is 1.45.

In relatively low or high blood pressure states, when the blood volume decreases or the body temperature of the sensor placement site decreases, b₃ takes 50% of a positive value. That is, if

$$\{ \begin{matrix}{d}_{6} \leq {k}_{sd - m - 0} < {d}_{6} + 0.1 \\ {k}_{d - m - a} \leq {c}_{4}\end{matrix} or \{ \begin{matrix}{d}_{8} < {k}_{sd - m - 2} < {d}_{7} \\ {k}_{d - m - a} \leq {c}_{4}\end{matrix} ,$$

then b₃=(c₄k_{d-m-a})×50/100;

In a normal blood pressure state, when the blood volume increases or the body temperature of the sensor placement site rises, b₃ takes 62% of a negative value. That is, if k_{sd-m-0}≥d₆+0.10 and k_{sd-m-2}≤d₈ and k_{d-m-a}≥c₅, then b₃=(c₅k_{d-m-a})×62/100;

In a state of relatively low or high blood pressure, when the blood volume increases or the body temperature of the sensor placement site increases, b₃ takes 45% of the negative value. That is, if

$$\{ \begin{matrix}{d}_{6} \leq {k}_{sd - m - 0} < {d}_{6} + 0.1 \\ {k}_{d - m - a} \geq {c}_{5}\end{matrix} or \{ \begin{matrix}{d}_{8} < {k}_{sd - m - 2} < {d}_{7} \\ {k}_{d - m - a} \geq {c}_{5}\end{matrix} ,$$

then b₃=(c₅k_{d-m-a})×45/100.

Fourth correction variable b₄:

The correction variables obtained in the step S4 further include a fourth correction variable b₄, which is used for correcting T_{d} in the case that the peripheral blood vessel dilation causes the lower limb blood pressure (relative to the radial artery blood pressure) to decrease. The applicable range of b₄ is b₄>0, and if b₄ is larger, the lower limb blood pressure is much lowered relative to the radial artery blood pressure.

Contraction and expansion of peripheral blood vessels may cause the peak of the toe pulse wave to move back and forth on a time axis. If tₘₐₓ₋ₜₒₑ≥t_{ch-toe}, then

$${k}_{s - t - t o e} = \frac{{t}_{max - toe} + {t}_{c h - t o e} + 400}{( {t}_{s - t o e} + 2 \; 00 ) ⨯ 2} ,$$

otherwise

$${k}_{s - t - toe} = \frac{{t}_{max - toe} + 200}{{t}_{s - toe} + 200} .$$

kₛ₋ₜ₋ₜₒₑ is the ratio of the time from a start point to a peak of the toe pulse wave to the time of the systole, and 200 is an adjustment coefficient. When the highest point of the peak moves back beyond the midpoint, that is, when tₘₐₓ₋ₜₒₑ≥t_{ch-toe}, kₛ₋ₜ₋ₜₒₑ is corrected; when the value of kₛ₋ₜ₋ₜₒₑ is large, indicating that the toe blood vessels dilate and the lower limb blood pressure decreases. That is, if kₛ₋ₜ₋ₜₒₑ>0.8, then b₄=kₛ₋ₜ₋ₜₒₑ−0.8. If kₛ₋ₜ₋ₜₒₑ≤0.8, b₄ is not applicable, then set b₄=0.

Fifth correction variable b₅;

The correction variables obtained in the step S4 further include a fifth correction variable b₅, the role and property of b₅ are the same as those of the b₄, and b₅ is used for correcting T_{d} in the case that the lower limb blood pressure decreases relative to the radial artery blood pressure.

$${k}_{s - t - toe} = \frac{{\int }_{0}^{{t}_{s - toe}} hdt}{{t}_{s - toe} {h}_{max - toe}} ,$$

kₛ₋ₘ₋ₜₒₑ is the ratio of the average height of the toe pulse wave systole to the maximum height hₘₐₓ₋ₜₒₑ; if kₛ₋ₘ₋ₜₒₑ is large, indicating that the toe pulse wave peak is broad and flat, suggesting that the toe blood vessels dilate, and the lower limb blood pressure decreases relative to the radial artery.

When the toe blood vessels do not dilate, b₅ is not applicable. That is, if kₛ₋ₘ₋ₜₒₑ<d₉, then set b₅=0. d₉=0.67 to 0.73, preferably is 0.7.

When the toe blood vessels dilate and the highest point of the pulse wave peak shifts backwards beyond the midpoint, b₅ takes a positive value. That is, if kₛ₋ₘ₋ₜₒₑ≥d₉ and kₛ₋ₜ₋ₜₒₑ≥0.8, then b₅=kₛ₋ₘ₋ₜₒₑ−d₉.

When the toe blood vessels dilate and the highest point of the pulse wave peak does not exceed the midpoint, b₅ takes half of the positive value. That is, if kₛ₋ₘ₋ₜₒₑ≥d₉ and kₛ₋ₜ₋ₜₒₑ<0.8, then b₅=(kₛ₋ₘ₋ₜₒₑ−d₉)/2.

Sixth correction variable b₆;

The correction variables obtained in the step S4 further include a sixth correction variable b₆, which represents a relative change in the area of two pulse waves, and is used for correcting T_{d} when the toe blood vessels dilate and the lower limb blood pressure decreases relative to the radial artery blood pressure. The applicable range of b₆ is b₆>0;

$${k}_{s - m - toe - ear} = \frac{{h}_{max} {\int }_{0}^{{t}_{s - toe}} hdt + ( {t}_{s} + {t}_{s - toe} ) \times 100}{{h}_{max - toe} {\int }_{0}^{{t}_{s}} hdt + ( {t}_{s} + {t}_{s - toe} ) \times 100} ,$$

kₛ₋ₘ₋ₜₒₑ-ear is the ratio of the area of the toe pulse wave systole to the area of the ear pulse wave systole, and 100 is the adjustment coefficient; kₛ₋ₘ₋ₜₒₑ-ear has the same role and property as those of kₜₛ₋ₜₒₑ₋ₑₐᵣ.

When the toe wave area is smaller than the ear wave area, the toe blood vessels have no relative dilation, and b₆ is not applicable. That is, if kₛ₋ₘ₋ₜₒₑ₋ₑₐᵣ<1.0, then set b₆=0.

Under the first precondition that the toe area is greatly larger than the ear area, toe blood vessels dilate more, and C₆ takes a constant of 1.08 as the maximum value for use. That is, if kₛ₋ₘ₋ₜₒₑ₋ₑₐᵣ>1.08, then set c₆=1.08.

If the shape of the ear pulse wave is normal, b₆ takes the maximum correction value. That is, if tₛ>220 and k_{sd-m-0}>0.88, then b₆=c₆−1.0.

If the ear pulse wave appears as a very sharp forward-inclined triangle or the waveform is very narrow, representing that the shape of the ear pulse wave is severely irregular. At this time, the relative change between two pulse waves is amplified, the correction value needs to be reduced for use, and b₆ takes ⅓ of the maximum correction value. That is, if tₛ<160 or k_{sd-m-0}<0.80, then b₆=(c₆−1.0)×0.34.

When the irregularity of the shape of the ear pulse wave is not too severe, b₆ takes ⅔ of the maximum correction value. That is, if 160<tₛ≤220 or 0.80<k_{sd-m-0}≤0.88, then b₆=(c₆−1.0)×0.67.

Under the second precondition that the toe area is larger than the ear area, relative dilatation of the toe blood vessels is not too severe, and c₆ takes a positive variable for use. That is, if 1.0≤kₛ₋ₘ₋ₜₒₑ₋ₑₐᵣ≤1.08, then c₆=kₛ₋ₘ₋ₜₒₑ₋ₑₐᵣ−1.0.

If the shape of the ear pulse wave is normal, b₆ takes a positive variable as the correction value. That is, if tₛ>220 and k_{sd-m-0}>0.88, then b₆=c₆.

If the shape of the ear pulse wave is severely irregular, the relative change between the two pulse waves is amplified, the correction value needs to be reduced for use, and b₆ takes ⅓ of a positive variable. That is, if tₛ≤160 or k_{sd-m-0}≤0.80, then b₆=c₆×0.34.

When the irregularity of the ear pulse wave is not too severe, b₆ takes ⅔ of a positive variable. That is, if 160<tₛ≤220 or 0.80<k_{sd-m-0}≤0.88, then b₆=c₆×0.67.

Seventh correction variable b₇;

The correction variables obtained in the step S4 further include a seventh correction variable b₇, the role and property of b₇ are the same as those of b₆, and b₇ represents the relative change of the systolic time of two pulse waves.

$${k}_{ts - toe - ear} = \frac{{t}_{s - toe} + 825}{{t}_{s} + 825} ,$$

kₜₛ₋ₜₒₑ₋ₑₐᵣ is the ratio of the time of systole on the toe pulse wave to the time of systole on the ear pulse wave, and 825 is the adjustment coefficient; increase in kₜₛ₋ₜₒₑ₋ₑₐᵣ suggests that the toe blood vessels dilate, and the lower limb blood pressure decreases relative to the radial artery blood pressure.

When the toe blood vessels have no relative dilation, b₇ is not applicable. That is, if kₜₛ₋ₜₒₑ₋ₑₐᵣ<1.0, then set b₇=0.

Under the first precondition that toe blood vessels have severely relative dilation comparing to radial blood vessels, c₇ takes a constant of 1.08 as the maximum value for use. That is, if kₜₛ₋ₜₒₑ₋ₑₐᵣ>1.08, then set c₇=1.08.

If the shape of the ear pulse wave is normal, b₇ takes the maximum correction value. That is, if tₛ>220 and k_{sd-m-0}>0.88, then b₇=c₇−1.0.

If the shape of the ear pulse wave is severely irregular, the relative change between the two pulse waves is amplified, the correction value needs to be reduced for use, and b₇ takes ⅓ of the maximum correction value. That is, if tₛ<160 or k_{sd-m-0}<0.80, then b₇=(c₇−1.0)×0.34.

If the irregularity of the shape of the ear pulse wave is not too severe, b₇ takes ⅔ of the maximum correction value. That is, if 160<tₛ≤220 or 0.80<k_{sd-m-0}≤0.88, then b₇=(c₇−1.0)×0.67.

Under the second precondition that the toe systolic time is greater than the ear systolic time, the relative dilatation of the toe blood vessels is not too severe comparing to that of radial blood vessels, and c₇ takes a positive variable for use. That is, if 1.0≤kₜₛ₋ₜₒₑ₋ₑₐᵣ≤1.08, then c₇=kₜₛ₋ₜₒₑ₋ₑₐᵣ−1.0

If the shape of the ear pulse wave is normal, b₇ takes a positive variable as the correction value. That is, if tₛ>220 and k_{sd-m-0}>0.88, then b₇=c₇.

If the irregularity of the shape of the ear pulse wave is too severe, b₇ takes ⅓ of a positive variable. That is, if tₛ≤160 or k_{sd-m-0}≤0.80, then b₇=c₇×0.34.

If the irregularity of the shape of the ear pulse wave is not too severe, b₇ takes ⅔ of the positive variable. That is, if 160<tₛ≤220 or 0.80<k_{sd-m-0}≤0.88, then b₇=c₇×0.67.

The total correction value in the step S5 is

$$B = {\sum }_{i = 1}^{7} {b}_{i} .$$

Where if bᵢ=0, indicating that bᵢ is not applicable. The step S6 is specifically: continuously acquiring the correction values in 8 cardiac cycles, and using the average value of the 8 values to overcome the disturbance of respiratory fluctuation, where the 8 values are selected by recursion, and the oldest matrix is eliminated each time when a new matrix is calculated. A correction method is: T_{dmb}=T_{dm}(1−Bₘ); where

$$B = \frac{1}{8} {\sum }_{i = 1}^{8} {B}_{i} , {T}_{d m} = \frac{1}{8} {\sum }_{i = 1}^{8} {T}_{di} ,$$

Bᵢ is the total correction value in the i-th cardiac cycle, and T_{di} is T_{d} in the i-th cardiac cycle.

### Embodiment 2

A method for correcting PTT associated with systolic blood pressure, includes the following steps:

S1) detecting a pulse wave at an ear in each cardiac cycle in real time and obtaining the following data: the height of an aortic valve closure point on the ear pulse wave denoted as h_{sd}, that is, the height at a junction between the systolic and diastolic phases on the ear pulse wave, the systolic time of the ear pulse wave denoted as tₛ, the diastolic time of the ear pulse wave denoted as t_{d}, and the maximum height of the ear pulse wave denoted as hₘₐₓ;

S2) detecting the pulse wave at a toe in each cardiac cycle in real time and obtaining the following data: the systolic time of the toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between the starting point to the midpoint of the peak of the toe pulse wave denoted as t_{ch-toe}, and the time interval between the starting point to the highest point of the peak of the toe pulse wave denoted as tₘₐₓ₋ₜₒₑ, where the midpoint of the peak refers to the midpoint of arising edge turning point and a falling edge turning point at the peak; the definition of the midpoint of the peak can be understood by referring to the literature YAN CHEN, CHANGYUN WEN, GUOCAI TAO, and MIN BI Continuous and Non-invasive Measurement of Systolic and Diastolic Blood Pressure by One Mathematical Model with the Same Model Parameters and Two Separate Pulse Wave Velocities.

S3) calculating the PTT associated with systolic blood pressure denoted as Tₛ, and the definition can be understood by referring to the literature YAN CHEN, CHANGYUN WEN, GUOCAI TAO, and MIN BI Continuous and Non-invasive Measurement of Systolic and Diastolic Blood Pressure by One Mathematical Model with the Same Model Parameters and Two Separate Pulse Wave Velocities; h is the amplitude of the ear pulse wave or the toe pulse wave in a longitudinal direction;

S4) by using the data in the same cardiac cycle acquired through step S1 and step S2, calculating a few correction variables a₁-win the cardiac cycle;

S5) according to the correction variables in the cardiac cycle acquired in step S4, calculating a total correction value in the cardiac cycle; and

S6) continuously acquiring the correction values in a plurality of cardiac cycles, and correcting the Tₛ acquired in the step S3.

By the method, the PTT associated with systolic blood pressure is determined by a time difference of an ear pulse wave and a toe pulse wave in the same cardiac cycle, and a few correction variables are extracted based on the pulse wave features, then a total correction value is acquired to perform adaptive correction on the irregular change of pulse wave transit time. The corrected transit time can be used with available mathematical models for continuously and accurately measuring systolic blood pressure in each cardiac cycle in a clinical setting.

First correction variable a₁:

The correction variables obtained in the step S4 include a first correction variable a₁, a₁ is used for correcting the type II changes in Tₛ in a hypotensive state, the applicable range of a₁ is a₁>0, and if a₁ is larger, the blood pressure is lower.

$${k}_{sd - m - 0} = \frac{{t}_{s} {h}_{sd}}{{\int }_{0}^{{t}_{s}} hdt} ,$$

k_{sd-m-0} represents the ratio of h_{sd} to the average height of the ear pulse wave. In some cases, under a hypotensive state, the pulse wave peak appears as a forward-inclined triangle. When h_{sd} decreases a lot, k_{sd-m-0} becomes smaller, indicating that the waveform at the end of the aortic systole is much lower, the continuous power for pushing wave transit is insufficient, and the transit time is prolonged. In this state, the diastolic information is unstable and should not be used. d₁=76 to 84, preferably is 80; d₁₋₂=104 to 112, preferably is 108.

When the continuous power for pushing pulse wave transit is insufficient, the transit time Tₛ is prolonged and needed be corrected by a₁. That is, if d₁≤k_{sd-m-0}≤d₁₋₂, then a₁=(d₁₋₂−k_{sd-m-0})×0.50;

When the continuous power for pushing pulse wave transit is seriously insufficient, the transit time Tₛ is prolonged a lot, and a₁ takes the upper limit value for correction. That is, if k_{sd-m-0}<d₁, then a₁=28×0.50;

When the continuous power for pushing pulse wave transit is sufficient, Tₛ does not need to be corrected, and a₁ is not applicable. That is, if k_{sd-m-0}>d₁₋₂, then a₁=0.

Second correction variable a₂:

The correction variables obtained in the step S4 also include a second correction variable a₂, a₂ is used for correcting the type II changes in Tₛ in a hypertensive state and in a change process from a normotensive state to the hypertensive state, the applicable range of a₂ is a₂>0, and if a₂ is larger, the systolic blood pressure is higher.

$${k}_{sd - m - ts} = \frac{{t}_{s} {h}_{sd}}{{\int }_{{t}_{s}}^{2 {t}_{s}} hdt} ,$$

k_{sd-m-ts} represents the ratio of h_{sd} to the average height of the tₛ to 2tₛ segments of the ear pulse wave diastole, and is used for determining the irregular change of the pulse wave diastole. For example, in the thoracoabdominal surgery, the upward pulling hook causes the aortic stress to change, so that the amplitude of the ear pulse wave diastole is reduced, and the k_{sd-m-ts} becomes larger.

$${k}_{sd - m - 2} = \frac{2 {t}_{s} {h}_{sd}}{{\int }_{{t}_{s}}^{2 {t}_{s}} hdt} ,$$

k_{sd-m-2} represents the ratio of h_{sd} to the average height of the ear pulse wave 0 to 2tₛ segments, includes the information of systolic and partial diastolic waveform, and is mainly used for a hypertensive state and a change process from a normotensive state to the hypertensive state, such as increase of heart rate and blood pressure caused by tracheal intubation. In the process of change from the normotensive state to the hypertensive state, the peak of the ear pulse wave gradually appears as an equilateral triangle or a backward-inclined triangle, h_{sd} gradually increases, and k_{sd-m-2} gradually becomes larger; in the hypertensive state, the entire ear pulse wave appears as an equilateral triangle or a backward-inclined triangle, h_{sd} rises a lot, and k_{sd-m-2} becomes very large; the peaks (i.e., the maximal blood pressure) of the triangles of the above two waveforms is very short in duration, the continuous power corresponding to the maximal blood pressure is insufficient, and the transit time Tₛ is relatively prolonged.

If |k_{sd-m-0}−k_{sd-m-ts}|≥40 and (k_{sd-m-0}+k_{sd-m-ts})/2≥k_{sd-m-2},

then k_{sd-m}=2×k_{sd-m-2}−(k_{sd-m-0}+k_{sd-m-ts})/2,

otherwise k_{sd-m}=k_{sd-m-2};

If the waveform of the ear pulse wave diastole has an irregular change, for example, if the upward pulling hook of the thoracoabdominal surgery causes the aortic stress to change, and the form of the pulse wave diastole changes significantly, the k_{sd-m} is corrected, otherwise k_{sd-m}=k_{sd-m-2}. d₂=1.17 to 1.27, preferably is1.22.

If k_{sd-m}>(d₂+(age-14)/15/100), where age is age, age≥14 years old, indicating that the entire ear pulse wave or the peak thereof becomes an equilateral triangle or a backward-inclined triangle, the continuous power corresponding to the maximal blood pressure is insufficient, the transit time Tₛ is relatively prolonged, and a₂ is needed for correction, then a₂=k_{sd-m}−(d₂+(age-14)/15/100).

If k_{sd-m}≥(d₂+(age-14)/15/100), the peak of the pulse wave is flat, the continuous power corresponding to the maximal blood pressure is sufficient, and a₂ is not needed for correction, then set a₂=0.

Third correction variable a₃:

The correction variables obtained in the step S4 further include a third correction variable a₃, which is used for correcting Tₛ in a state that the blood volume changes or the body temperature of a sensor placement site changes.

$${k}_{d - m - {t}_{d}} = \frac{{\int }_{{t}_{s}}^{{t}_{s} + {t}_{d}} hdt}{{t}_{d} {h}_{max}} ,$$

k_{d-m-t}_{_{d}} is the ratio of the average height of the ear pulse wave diastole to the maximum height hₘₐₓ. The blood volume decreases when a patient fasts and drinks less water before surgery, k_{d-m-t}_{_{d}} decreases, the transit time is prolonged, when the blood volume increases due to blood transfusion and IV transfusion in the operation, k_{d-m-t}_{_{d}} increases, and the transit time is shortened.

If k_{sd-m-ts}≤d₃₋₂, indicating that the early diastole of the ear pulse wave rises and exceeds a normal range, k_{d-m-t}_{_{d}} needs to be corrected, and the correction result is noted as k_{d-m-t}_{_{d}}₋₁.

k_{d-m-t}_{_{d}}₋₁=k_{d-m-t}_{_{d}}−(d₃₋₂−k_{sd-m-ts})×75/100; if k_{d-m-t}_{_{d}}≤d₃, indicated that the ear pulse wave is disturbed, then k_{d-m-t}_{_{d}}=d₃. d₃=0.02 to 0.14, preferably is 0.08; d₃₋₂=1.21 to 1.31, preferably is 1.26.

$${k}_{d - m - {t}_{d - toe}} = \frac{{\int }_{{t}_{s - toe}}^{{t}_{s - toe} + {t}_{d - toe}} hdt}{{t}_{d - toe} {h}_{max - toe}} ,$$

k_{d-m-t}_{_{d-toe}} is the ratio of the average height of the toe pulse wave diastole to the maximum height hₘₐₓ₋ₜₒₑ, tₛ₋ₜₒₑ represents the systolic time of the toe pulse wave, and t_{d-toe} represents the diastolic time of the toe pulse wave. If k_{d-m-t}_{_{d-toe}}≤d₃, then k_{d-m-t}_{_{d-toe}}=d₃. k_{d-m-t}_{_{d-toe}} and k_{d-m-t}_{_{d}} have the same role and property.

k_{d-m-a}=(k_{d-m-t}_{_{d}}₋₁+k_{d-m-t}_{_{d-toe}})/2 two variables from the ear and toe pulse waves with the same property are combined, and the average value is taken as the variable for correcting Tₛ. If the pulse wave diastole has an irregular change, k_{d-m-a} is corrected.

If |k_{sd-m-0}−k_{sd-m-ts}|≥40 and (k_{sd-m-0}+k_{sd-m-ts})/2≥k_{sd-m-2} and k_{sd-m-ts}≥d₃₋₂,

then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2.

In the state that the blood volume is normal and the body temperature of the sensor placement site is also normal, a₃ is not applicable. That is, if c₄<k_{d-m-a}<c₅, then set a₃=0. c₄=(d₄+(age-14)/8)/100, d₄=23 to 35, preferably is 29; c₅=(d₅+(age-14)/8)/100, d₅=27 to 39, preferably is33.

In extremely low or high blood pressure states, the information of diastolic period is unstable, and a₃ is not applicable. That is, if k_{sd-m-0}<d₆ or k_{sd-m-2}>d₇, then set a₃=0. d₆=0.97 to 1.03, preferably is1.00; d₇=1.52 to 1.58, preferably is 1.55.

In a normal blood pressure state, when the blood volume decreases or the body temperature of the sensor placement site decreases, a₃ takes 67% of a positive value. That is, if k_{sd-m-0}≥d₆+0.10 and k_{sd-m-2}≤d₈ and k_{d-m-a}≤c₄, then a₃=(c₄−k_{d-m-a})×67/100. d₈=1.42 to 1.48, preferably is 1.45.

In relatively low or high blood pressure states, when the blood volume decreases or the body temperature of the sensor placement site decreases, a₃ takes 50% of a positive value. That is, if

$$\{ \begin{matrix}{d}_{6} \leq {k}_{sd - m - 0} < {d}_{6} + 0.1 \\ {k}_{d - m - a} \leq {c}_{4}\end{matrix} or$$

$$\{ \begin{matrix}{d}_{8} \leq {k}_{sd - m - 2} < {d}_{7} \\ {k}_{d - m - a} \leq {c}_{4}\end{matrix} ,$$

then a₃=(c₄−k_{d-m-a})×50/100;

In a normal blood pressure state, when the blood volume increases or the body temperature of the sensor placement site increases, a₃ takes 62% of a negative value. That is, if k_{sd-m-0}≥d₆+0.10 and k_{sd-m-2}≤d₁₈ and k_{d-m-a}≥c₅, then a₃=(c₅−k_{d-m-a})×62/100;

In a state of relatively low or high blood pressure, when the blood volume increases or the body temperature of the sensor placement site increases, a₃ takes 45% of the negative value. That is, if

$$\{ \begin{matrix}{d}_{6} \leq {k}_{sd - m - 0} < {d}_{6} + 0.1 \\ {k}_{d - m - a} \geq {c}_{5}\end{matrix} or$$

$$\{ \begin{matrix}{d}_{8} < {k}_{sd - m - 2} < {d}_{7} \\ {k}_{d - m - a} \geq {c}_{5}\end{matrix} ,$$

then a₃=(c₅k_{d-m-a)}×45/100.

Fourth correction variable a₄:

The correction variables obtained in the step S4 further include a fourth correction variable a₄, which is used for correcting Tₛ in the case that the peripheral blood vessel dilation causes the lower limb blood pressure (relative to the radial artery blood pressure) to decrease. The applicable range of a₄ is a₄22 0, and if a₄ is greater, the lower limb blood pressure is much lowered relative to the radial artery blood pressure.

Contraction and expansion of peripheral blood vessels may cause the peak of the toe pulse wave to move back and forth on a time axis. If tₘₐₓ₋ₜₒₑ≥t_{ch-toe}, then

$${k}_{s - t - toe} = \frac{{t}_{max - toe} + {t}_{ch - toe} + 400}{( {t}_{s - toe} + 200 ) \times 2} ,$$

otherwise

$${k}_{s - t - toe} = \frac{{t}_{max - toe} + 200}{{t}_{s - toe} + 200} .$$

kₛ₋ₜ₋ₜₒₑ is the ratio of the time from the start point to the peak of the toe pulse wave to the time of the systole, and 200 is an adjustment coefficient. When the highest point of the peak moves back beyond the midpoint, that is, when tₘₐₓ₋ₜₒₑ≥t_{ch-toe}, kₛ₋ₜ₋ₜₒₑ is corrected; when the value of kₛ₋ₜ₋ₜₒₑ is relatively large, indicating that the toe blood vessels dilate and the lower limb blood pressure decreases. That is, if kₛ₋ₜ₋ₜₒₑ>0.8, then a₄=kₛ₋ₜ₋ₜₒₑ-0.8. If kₛ₋ₜ₋ₜₒₑ≤0.8, a₄ is not applicable, then set a₄=0.

Fifth correction variable a₅;

The correction variables obtained in the step S4 further include a fifth correction variable a₅, the role and property acts are the same as those of the a₄, and a₅ is used for correcting Tₛ in the case that the lower limb blood pressure decreases relative to the radial artery blood pressure.

$${k}_{s - m - toe} = \frac{{\int }_{0}^{{t}_{s - toe}} h dt}{{t}_{s - toe} {h}_{max - toe}} ,$$

kₛ₋ₘ₋ₜₒₑ is the ratio of the average height of the toe pulse wave systole to the maximum height hₘₐₓ₋ₜₒₑ; if the kₛ₋ₘ₋ₜₒₑ is large, indicating that the toe pulse wave peak is broad and flat, suggesting that the toe blood vessels dilate, and the lower limb blood pressure decreases relative to the radial artery. a₅ has the same action and property as those of a₄.

When the toe blood vessels do not dilate, as is not applicable. That is, if kₛ₋ₘ₋ₜₒₑ<d₉, then set a₅=0. d_{9=0.67} to 0.73, preferably is 0.7.

When the toe blood vessels dilate and the highest point of the pulse wave peak shifts backward beyond the midpoint, as takes a positive value. That is, if kₛ₋ₘ₋ₜₒₑ≥d₉ and kₛ₋ₜ₋ₜₒₑ≥0.8, then a₅=kₛ₋ₘ₋ₜₒₑ−d₉.

When the toe blood vessels dilate and the highest point of the pulse wave peak does not exceed the midpoint, as takes half of the positive value. That is, if kₛ₋ₘ₋ₜₒₑ≥d₉ and kₛ₋ₜ₋ₜₒₑ<0.8, then a₅=(kₛ₋ₘ₋ₜₒₑ−d₉)/2.

Sixth correction variable a₆;

The correction variables obtained in the step S4 further include a sixth correction variable a₆, which represents a relative change in the area of two pulse waves, and is used for correcting Tₛ when the toe blood vessels dilate and the lower limb blood pressure decreases relative to the radial artery blood pressure. The applicable range of a₆ is a₆>0.

$${k}_{s - m - toe - ear} = \frac{{h}_{max} {\int }_{0}^{{t}_{s - toe}} h dt + ( {t}_{s} + {t}_{s - toe} ) \times 100}{{h}_{max - toe} {\int }_{0}^{{t}_{s}} hdt + ( {t}_{s} + {t}_{s - toe} ) \times 100} ,$$

kₛ₋ₘ₋ₜₒₑ₋ₑₐᵣ is the ratio of the area of the toe pulse wave systole to the area of the ear pulse wave systole, and 100 is the adjustment coefficient; kₛ₋ₘ₋ₜₒₑ-ear has the same role and property as those of kₜₛ₋ₜₒₑ₋ₑₐᵣ.

When the toe wave area is smaller than the ear wave area, the toe blood vessels have no relative dilation, and a₆ is not applicable. That is, if kₛ₋ₘ₋ₜₒₑ₋ₑₐᵣ<1.0, then set a₆=0.

Under the first precondition that the toe area is larger than the ear area, toe blood vessels dilate more, and c₆ takes a constant of 1.08 as the maximum value for use. That is, if kₛ₋ₘ₋ₜₒₑ₋ₑₐᵣ>1.08, then set c₆=1.08.

If the shape of the ear pulse wave is normal, a₆ takes the maximum correction value. That is, if tₛ>220 and k_{sd-m-0}>0.88, then a₆=c₆−1.0.

If the ear pulse wave appears as a very sharp forward-inclined triangle or the waveform is very narrow, indicating that the ear pulse waveform is severely irregular. At this time, the relative change between the two pulse waves is amplified, the correction value needs to be reduced for use, and a₆ takes ⅓ of the maximum correction value. That is, if tₛ<160 or k_{sd-m-0}<0.80, then a₆=(c₆−1.0)×0.34.

When the irregularity of the shape of the ear pulse wave is not too severe, a₆ takes ⅔ of the maximum correction value. That is, if 160<tₛ≤220 or 0.80<k_{sd-m-0}≤0.88, then a₆=(c₆−1.0)×0.67.

Under the second precondition that the toe area is larger than the ear area, the relative dilatation of the toe blood vessels is not too severe, and c₆ takes a positive variable for use. That is, if 1.0≤kₛ₋ₘ₋ₜₒₑ₋ₑₐᵣ≤1.08, then c₆=kₛ₋ₘ₋ₜₒₑ₋ₑₐᵣ−1.0.

If the shape of the ear pulse wave is normal, a₆ takes a positive variable as the correction value. That is, if tₛ>220 and k_{sd-m-0}>0.88, then a₆=c₆.

If the shape of the ear pulse wave is severely irregular, the relative change between the two pulse waves is amplified, the correction value needs to be reduced for use, and a₆ takes ⅓ of the positive variable. That is, if tₛ≤160 or k_{sd-m-0}≤0.80, then a₆=c₆×0.34.

When the irregularity of the ear pulse wave is not too severe, a₆ takes ⅔ of the positive variable. That is, if 160<tₛ≤220 or 0.80<k_{sd-m-0}≤0.88, then a₆=c₆×0.67.

Seventh correction variable a₇;

The correction variables obtained in the step S4 further include a seventh correction variable a₇, the role and property of a₇ are the same as those of a₆, and a₇ represents the relative change of the systolic time of two pulse waves.

$${k}_{ts - toe - ear} = \frac{{t}_{s - toe} + 825}{{t}_{s} + 825} ,$$

kₜₛ₋ₜₒₑ₋ₑₐᵣ is the ratio of the time of systole on the toe pulse wave to the time of the systole on the ear pulse wave, and 825 is an adjustment coefficient; increase in kₜₛ₋ₜₒₑ₋ₑₐᵣ suggests that the toe blood vessels dilate, and the lower limb blood pressure decreases relative to the radial artery blood pressure.

When the toe blood vessels have no relative dilation, a₇ is not applicable. That is, if kₜₛ₋ₜₒₑ₋ₑₐᵣ<1.0, then set a₇=0.

Under the first precondition that the toe blood vessels have severely relative dilation comparing to radial blood vessels, c₇ takes a constant of 1.08 as the maximum value for use. That is, if kₜₛ₋ₜₒₑ₋ₑₐᵣ>1.08, then set c₇=1.08.

If the form of the ear pulse wave is normal, a₇ takes the maximum correction value. That is, if tₛ>220 and k_{sd-m-0}>0.88, then a₇=c₇−1.0.

If the shape of the ear pulse wave is severely irregular, the relative change between the two pulse waves is amplified, the correction value needs to be reduced for use, and a₇ takes ⅓ of the maximum correction value. That is, if tₛ<160 or k_{sd-m-0}<0.80, then a₇=(c₇−1.0)×0.34.

If the irregularity of the shape of the ear pulse wave is not too severe, a₇ takes ⅔ of the maximum correction value. That is, if 160<tₛ≤220 or 0.80<k_{sd-m-0}≤0.88, then a₇=(c₇−1.0)×0.67.

Under the second precondition that the toe systolic time is greater than the ear systolic time, the relative dilatation of the toe blood vessels is not too severe comparing to that of radial blood vessels, and c₇ takes a positive variable for use. That is, if 1.0≤kₜₛ₋ₜₒₑ₋ₑₐᵣ≤1.08, then c₇=kₜₛ₋ₜₒₑ₋ₑₐᵣ−1.0.

If the shape of the ear pulse wave is normal, a₇ takes a positive variable as the correction value. That is, if tₛ>220 and k_{sd-m-0}>0.88, then a₇=c₇.

If the irregularity of the shape of the ear pulse wave is too severe, a₇ takes ⅓ of the positive variable. That is, if tₛ≤160 or k_{sd-m-0}≤0.80, then a₇=c₇×0.34.

If the irregularity of the shape of the ear pulse wave is not too severe, a₇ takes ⅔ of the positive variable. That is, if 160<tₛ≤220 or 0.80<k_{sd-m-0}≤0.88, then a₇=c₇×0.67.

The total correction value in the step S5 is

$${A}_{m} = \frac{1}{8} {\sum }_{i = 1}^{8} {A}_{i} , {T}_{sm} = \frac{1}{8} {\sum }_{i = 1}^{8} {T}_{si} ,$$

where A is the sum of the correction variables a₁-a₇, where if aᵢ=0, indicating that aᵢ is not applicable. The step S6 is specifically: continuously acquiring the correction values in 8 cardiac cycles, and using the average value of the 8 values to overcome the disturbance of respiratory fluctuation, where the 8 values are selected by recursion, and the oldest matrix is eliminated each time when a new matrix is calculated. The correction method is: Tₛₘₐ=Tₛₘ(1−Am); where

$$A = {\sum }_{i = 1}^{7} {a}_{i} ,$$

in which Tₛₘₐ is the Tₛ after correction, Tₛₘ is the averaged Tₛ in 8 cardiac cycles, Amis the averaged A in 8 cardiac cycles, Aᵢ is the total correction value in the i-th cardiac cycle, and Tₛᵢ is Tₛ in the i-th cardiac cycle.

Finally, it should be noted that the above embodiments are only used for illustrating the technical solution of the present invention, rather than limiting the present invention. Although the present invention has been described in detail with reference to the foregoing embodiments, those of ordinary skill in the art will appreciate that the technical solutions described in the foregoing embodiments can still be modified, or some or all the technical features can be equivalently replaced. These modifications or replacement do not make the essence of the corresponding technical solution detract from the scope of the technical solutions of the embodiments of the present invention, and are intended to be included within the scope of the claims and the description of the present invention.

What is claimed is:

1. A method for correcting pulse wave transit time associated with diastolic blood pressure, characterized in that, comprising the following steps:
S1) detecting a pulse wave at an ear in each cardiac cycle in real time and obtaining the following data: the height of an aortic valve closure point on an ear pulse wave denoted as h_{sd}, the systolic time of the ear pulse wave denoted as tₛ, the diastolic time of the ear pulse wave denoted as t_{d}, and the maximum height of the ear pulse wave denoted as hₘₐₓ;
S2) detecting the pulse wave at a toe in each cardiac cycle in real time and obtaining the following data: the systolic time of a toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between the starting point of the toe pulse wave and the midpoint of the systolic peak of the toe pulse wave denoted as t_{ch-toe}, the time interval between the starting point of the toe pulse wave and the highest point of the systolic peak of the toe pulse wave denoted as tₘₐₓ₋ₜₒₑ, wherein the midpoint of the peak refers to the midpoint of arising edge turning point and a falling edge turning point at the peak;
h_{sd} refers to the amplitude of the aortic valve closure point of the ear pulse wave relative to the starting point in a cardiac cycle;
hₘₐₓ refers to the maximum amplitude of the systolic peak of the ear pulse wave in a cardiac cycle;
hₘₐₓ₋ₜₒₑ refers to the maximum amplitude of the systolic peak of the toe pulse wave in a cardiac cycle;
tₛ (the systolic time of the ear pulse wave) refers to the time difference between the starting point and the aortic valve closure point on the ear pulse wave in a cardiac cycle;
t_{d} (the diastolic time of the ear pulse wave)refers to the time difference between the aortic valve closure point of the ear pulse wave in one cardiac cycle and the starting point of the ear pulse wave in the next cardiac cycle;
tₛ₋ₜₒₑ (the systolic time of the toe pulse wave) refers to the time difference between the starting point and the aortic valve closure point on the toe pulse wave in a cardiac cycle;
t_{d-toe} (the diastolic time of the toe pulse wave) refers to the time difference between the aortic valve closure point of the toe pulse wave in one cardiac cycle and the starting point of the toe pulse wave in the next cardiac cycle;
S3) calculating the pulse wave transit time associated with diastolic blood pressure denoted as T_{d}, wherein T_{d} refers to time difference between the starting point of the ear pulse wave and the starting point of the toe pulse wave, and h is the amplitude of the ear pulse wave or the toe pulse wave in a longitudinal direction, As for the pulse wave in plane coordinates, the ordinate is amplitude h, the abscissa is time t, and the pulse wave starting point is the coordinate origin, h refers to the height of a specific point on the pulse wave of the ear or toe, not just one of the toe pulse wave or ear pulse wave; h is an unknown quantity, which means the amplitude of the pulse wave at any moment;
S4) by using the data in the same cardiac cycle acquired through the step S1 and the step S2, calculating a plurality of correction variables b₁-b₇ in the cardiac cycle;
S5) according to the correction variables in the cardiac cycle acquired in the step S4, calculating a total correction value in the cardiac cycle; and
S6) continuously acquiring the correction values in a plurality of cardiac cycles, and correcting the Ta acquired in the step S3.

2. The method for correcting pulse wave transit time associated with diastolic blood pressure according to claim 1, characterized in that, the total correction value in the step S5 is
$$B = {\sum }_{i - 1}^{7} {b}_{i} ;$$
where B is the sum of the correction variables b₁-b₇,bᵢ is the i-th correction variable.

3. The method for correcting pulse wave transit time associated with diastolic blood pressure according to claim 1, characterized in that, in the step S6, the correction values in 8 cardiac cycles are continuously acquired; the corrected value of T_{d} is T_{dmb}, which is calculated by T_{dmb}=T_{dm}(1−Bₘ), where T_{dm} is the average value of T_{d} for 8 cardiac cycles
$${T}_{d m} = \frac{1}{8} {\sum }_{i = 1}^{8} {T}_{di} ,$$
T_{di} is the T_{d} in the i-th cardiac cycle; Bₘ is the average of the total correction value of 8 cardiac cycles, Bᵢ is the total correction value under the i-th cardiac cycle, wherein
$${B}_{m} = \frac{1}{8} {\sum }_{i = 1}^{8} {B}_{i} , {T}_{d m} = \frac{1}{8} {\sum }_{i = 1}^{8} {T}_{di} ,$$
Bᵢ is the total correction value in the i-th cardiac cycle, and T_{di} is T_{d} in the i-th cardiac cycle.

4. The method for correcting pulse wave transit time associated with diastolic blood pressure according to claim 1, characterized in that, the first correction variable b₁ is calculated by the following formulas:
if d_{1-b}≤k_{sd-m-0}≤d_{1-2-b}, then b₁=(d_{1-2-b}−k_{sd-m-0})×0.4; if k_{sd-m-0}<d_{1-b}, then b₁=24×0.4; if k_{sd-m-0}>d_{1-2-b}, then b₁=0; wherein d_{1-b}=74 to 82, d_{1-2-b}=98 to 106, and
$${k}_{sd - m - 0} = \frac{{t}_{s} {h}_{sd}}{{\int }_{0}^{{t}_{s}} hdt} .$$

5. The method for correcting pulse wave transit time associated with diastolic blood pressure according to claim 1, characterized in that, the second correction variable b₂ is calculated by the following formulas:
if k_{sd-m}>(d_{2-b}+(age-14)/15/100), then b₂=(k_{sd-m}−(d_{2-b}+(age-14)/15/100))×0.5;
if k_{sd-m}≤(d_{2-b}+(age-14)/15/100), then b₂=0;
wherein d_{2-b}=1.33 to 1.43, age is age, if |k_{sd-m-0}−k_{sd-m-ts}|≥40 and (k_{sd-m-0}+k_{sd-m-ts})/2≥k_{sd-m-2}, then k_{sd-m}=2×k_{sd-m-2}−(k_{sd-m-0}+k_{sd-ts})/2, otherwise k_{sd-m}=k_{sd-m-2};
$${k}_{sd - m - 0} = \frac{{t}_{s} {h}_{sd}}{{\int }_{0}^{{t}_{s}} hdt} , {k}_{sd - m - ts} = \frac{{t}_{s} {h}_{sd}}{{\int }_{{t}_{s}}^{2 {t}_{s}} hdt} , {k}_{sd - m - 2} = \frac{2 {t}_{s} {h}_{sd}}{{\int }_{0}^{2 {t}_{s}} hdt} .$$

6. The method for correcting pulse wave transit time associated with diastolic blood pressure according to claim 1, characterized in that, the third correction variable b₃ is calculated by the following formulas:
if c₄<k_{d-m-a}<c₅, then b₃=0;
if k_{sd-m-0}<d₆ or k_{sd-m-2}>d₇, then b₃=0;
if k_{sd-m-0}≥d₆+0.10, k_{sd-m-2}≤d₈ and k_{d-m-a}≤c₄, then b₃=(c₄−k_{d-m-a})×67/100;
if
$$\{ \begin{matrix}{d}_{6} \leq {k}_{sd - m - 0} < {d}_{6} + 0.1 \\ {k}_{d - m - a} \leq {c}_{4}\end{matrix} or$$
$$\{ \begin{matrix}{d}_{8} < {k}_{sd - m - 2} < {d}_{7} \\ {k}_{d - m - a} \leq {c}_{4}\end{matrix} ,$$
then b₃=(c₄−k_{d-m-a})×50/100;
if k_{sd-m-0}≥d₆+0.10 and k_{sd-m-2}≤d₈ and k_{d-m-a}≥c₅, then b₃=(c₅−k_{d-m-a})×62/100;
if
$$\{ \begin{matrix}{d}_{6} \leq {k}_{sd - m - 0} < {d}_{6} + 0.1 \\ {k}_{d - m - a} \geq {c}_{5}\end{matrix} or$$
$$\{ \begin{matrix}{d}_{8} < {k}_{sd - m - 2} < {d}_{7} \\ {k}_{d - m - a} \geq {c}_{5}\end{matrix} ,$$
then b₃=(c₅=k_{d-m-a})×45/100;
wherein if |k_{sd-m-0}−k_{sd-m-ts}|≥40 and (k_{sd-m-0}+k_{sd-m-ts})/2≥k_{sd-m-2} and k_{sd-m-ts}≥d₃₋₂, then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2, otherwise k_{d-m-a}=(k_{d-m-t}_{_{d}}₋₁+k_{d-m-t}_{_{d-toe}})/2;
if k_{sd-m-ts}≤d₃₋₂, then k_{d-m-t}_{_{d}}₋₁=k_{d-m-t}_{_{d}}−(d₃₋₂−k_{sd-m-ts})×75/100; if k_{d-m-t}_{_{d}}≤d₃, then k_{d-m-t}_{_{d}}₋₁=d₃; if k_{d-m-t}_{_{d-toe}}≤d₃, then k_{d-m-t}_{_{d-toe}}=d₃;
$${k}_{d - m - {t}_{d}} = \frac{{\int }_{{t}_{s}}^{{t}_{s} + {t}_{d}} hdt}{{t}_{d} {h}_{max}} , {k}_{sd - m - 0} = \frac{{t}_{s} {h}_{sd}}{{\int }_{0}^{{t}_{s}} hdt} , {k}_{sd - m - 2} = \frac{2 {t}_{s} {h}_{sd}}{{\int }_{0}^{2 {t}_{s}} hdt} ,$$
$${k}_{sd - m - ts} = \frac{{t}_{s} {h}_{sd}}{{\int }_{{t}_{s}}^{2 {t}_{s}} hdt} , {k}_{d - m - {t}_{d - toe}} = \frac{{\int }_{{t}_{s - toe}}^{{t}_{s toe} + {t}_{d toe}} hdt}{{t}_{d - toe} {h}_{max - toe}} ;$$
and
c₄=(d₄+(age-14)/8)/100, d₄=23 to 35, c₅=(d₅+(age-14)/8)/100, d₅=27 to 39, d₆=0.97 to 1.03, d₇=1.52 to 1.58, d₈=1.42 to 1.48, d₃₋₂=1.21 to 1.31, d₃=0.02 to 0.14, and age is age.

7. The method for correcting pulse wave transit time associated with diastolic blood pressure according to claim 1, characterized in that, the fourth correction variable b₄ is calculated by the following formulas:
if kₛ₋ₜ₋ₜₒₑ>0.8, then b₄=kₛ₋ₜ₋ₜₒₑ−0.8;
if kₛ₋ₜ₋ₜₒₑ≤0.8, then b₄=0;
wherein if tₘₐₓ₋ₜₒₑ≥t_{ch-toe}, then
$${k}_{s - t - toe} = \frac{{t}_{max - toe} + {t}_{ch - toe} + 400}{( {t}_{s - toe} + 200 ) \times 2} ,$$
otherwise
$${k}_{s - t - toe} = \frac{{t}_{max - toe} + 200}{{t}_{s - toe} + 200} .$$

8. The method for correcting pulse wave transit time associated with diastolic blood pressure according to claim 1, characterized in that, the fifth correction variable b₅ is calculated by the following formulas:
if kₛ₋ₘ₋ₜₒₑ<d₉, then b₅=0;
if kₛ₋ₘ₋ₜₒₑ≥d₉ and kₛ₋ₜ₋ₜₒₑ≥0.8, then b₅=kₛ₋ₘ₋ₜₒₑ−d₉;
if kₛ₋ₘ₋ₜₒₑ≥d₉ and kₛ₋ₜ₋ₜₒₑ<0.8, then b₅=(kₛ₋ₘ₋ₜₒₑ−d₉)/2;
wherein d₉=0.67 to 0.73, and
$${k}_{s - m - toe} = \frac{{\int }_{0}^{{t}_{s - toe}} h dt}{{t}_{s - toe} {h}_{max - toe}} .$$

9. The method for correcting pulse wave transit time associated with diastolic blood pressure according to claim 1, characterized in that, the six correction variable b₆ is calculated by the following formulas:
if kₛ₋ₘ₋ₜₒₑ₋ₑₐᵣ<1.0, then b₆=0;
when kₛ₋ₘ₋ₜₒₑ₋ₑₐᵣ>1.08, then c₆=1.08, meantime, if tₛ>220 and k_{sd-m-0}>0.88, then b₆=c₆−1.0, if tₛ<160 or k_{sd-m-0}<0.80, then b₆=(c₆−1.0)×0.34, if 160<tₛ≤220 or 0.80<k_{sd-m-0}≤0.88, then b₆=(c₆−1.0)×0.67;
when 1.0≤kₛ₋ₘ₋ₜₒₑ₋ₑₐᵣ≤1.08, then c₆=kₛ₋ₘ₋ₜₒₑ₋ₑₐᵣ−1.0, meantime, if tₛ>220 and k_{sd-m-0}≥0.88, then b₆=c₆, if tₛ≤160 or k_{sd-m-0}≤0.80, then b₆=c₆×0.34, if 160<tₛ≤220 or 0.80<k_{sd-m-0}≤0.88, then b₆=c₆×0.67;
wherein
$${k}_{s - m - toe - ear} = \frac{{h}_{max} {\int }_{0}^{{t}_{s - toe}} h dt + ( {t}_{s} + {t}_{s - toe} ) \times 100}{{h}_{max - toe} {\int }_{0}^{{t}_{s}} h dt + ( {t}_{s} + {t}_{s - toe} ) \times 100} , and$$
$${k}_{sd - m - 0} = \frac{{t}_{s} {h}_{sd}}{{\int }_{0}^{{t}_{s}} h dt} .$$

10. The method for correcting pulse wave transit time associated with diastolic blood pressure according to claim 1, characterized in that, the seventh correction variable b₇ is calculated by the following formulas:
if kₜₛ₋ₜₒₑ₋ₑₐᵣ<1.0, then b₇=0;
when kₜₛ₋ₜₒₑ₋ₑₐᵣ>1.08, then c₇=1.08, meantime, if tₛ>220 and k_{sd-m-0}>0.88, then b₇=c₇−1.0, if tₛ<160 or k_{sd-m-0}<0.80, then b₇=(c₇−1.0)×0.34, if 160<tₛ≤220 or 0.80<k_{sd-m-0}≤0.88, then b₇=(c₇−1.0)×0.67;
when 1.0≤kₜₛ₋ₜₒₑ₋ₑₐᵣ≤1.08, then c₇=kₜₛ₋ₜₒₑ₋ₑₐᵣ−1.0, meantime, if tₛ>220 and k_{sd-m-0}>0.88, then b₇=c₇, if tₛ≤160 or k_{sd-m-0}≤0.80, then b₇=c₇×0.34, if 160<tₛ≤220 or 0.80<k_{sd-m-0}≤0.88, then b₇=c₇×0.67;
wherein
$${k}_{ts - toe - ear} = \frac{{t}_{s - toe} + 825}{{t}_{s} + 825} , and {k}_{sd - m - 0} = \frac{{t}_{s} {h}_{sd}}{{\int }_{0}^{{t}_{s}} h dt} .$$

11. A method for correcting pulse wave transit time associated with systolic blood pressure, characterized in that, comprising the following steps:
S1) detecting a pulse wave at an ear in each cardiac cycle in real time and obtaining following data: the height of an aortic valve closure point on an ear pulse wave denoted as h_{sd}, the systolic time of the ear pulse wave denoted as tₛ, the diastolic time of the ear pulse wave denoted as t_{d}, and the maximum height of the ear pulse wave denoted as hₘₐₓ;
S2) detecting the pulse wave at a toe in each cardiac cycle in real time and obtaining the following data: the systolic time of a toe pulse wave denoted as tₛ₋ₜₒₑ, the diastolic time of the toe pulse wave denoted as t_{d-toe}, the maximum height of the toe pulse wave denoted as hₘₐₓ₋ₜₒₑ, the time interval between starting point to a midpoint of a peak of the toe pulse wave denoted as t_{ch-toe}, and the time interval between the starting point to the highest point of the peak of the toe pulse wave denoted as tₘₐₓ₋ₜₒₑ, wherein the midpoint of the peak refers to the midpoint of arising edge turning point and a falling edge turning point at the peak;
S3) calculating the pulse wave transit time associated with systolic blood pressure denoted as Tₛ, wherein Tₛ refers to a time difference between the aortic valve closure point on the ear pulse wave and the aortic valve closure point on the toe pulse wave, and h is the amplitude of the ear pulse wave or the toe pulse wave in a longitudinal direction;
S4) by using the data in the same cardiac cycle acquired through the step S1 and the step S2, calculating a plurality of correction variables a₁-a₇ the cardiac cycle;
S5) according to the correction variables in the cardiac cycle acquired in the step S4, calculating a total correction value in the cardiac cycle; and
S6) continuously acquiring the correction values in a plurality of cardiac cycles, and correcting the Tₛ acquired in the step S3.

12. The method for correcting pulse wave transit time associated with systolic blood pressure according to claim 11, characterized in that, the total correction value in the step S5 is
$$A = {\sum }_{i = 1}^{7} {a}_{i} ,$$
where A is the sum of the correction variables a₁-a₇,aᵢ is the i-th correction variable.

13. The method for correcting pulse wave transit time associated with systolic blood pressure according to claim 11, characterized in that, in the step S6, the correction values in 8 cardiac cycles are continuously acquired; the correction method is: Tₛₘₐ=Tₛₘ(1−Am); wherein
$${A}_{m} = \frac{1}{8} {\sum }_{i = 1}^{8} {A}_{i} , {T}_{sm} = \frac{1}{8} {\sum }_{i = 1}^{8} {T}_{si} ,$$
in which Tₛₘₐ is the Tₛ after correction, Tₛₘ is the averaged Tₛ in 8 cardiac cycles, Amis the averaged A in 8 cardiac cycles, Aᵢ is the total correction value in the i-th cardiac cycle, and Tₛᵢ is Tₛ in the i-th cardiac cycle.

14. The method for correcting pulse wave transit time associated with systolic blood pressure according to claim 11, characterized in that, the first correction variable a₁ is calculated by the following formulas:
if d₁≤k_{sd-m-0}≤d₁₋₂, then a₁=(d₁₋₂−k_{sd-m-0})×0.50; if k_{sd-m-0}<d₁, then a₁=28×0.50; and
if k_{sd-m-0}>d₁₋₂, then a₁=0; wherein
$${k}_{sd = m - 0} = \frac{{t}_{s} {h}_{sd}}{{\int }_{0}^{{t}_{s}} h dt} ,$$
d₁=76 to 84, and d₁₋₂=104 to 112.

15. The method for correcting pulse wave transit time associated with systolic blood pressure according to claim 11, characterized in that, the second correction variable a₂ is calculated by the following formulas:
if k_{sd-m}>(d₂+(age-14)/15/100), then a₂=k_{sd-m}−(d₂+(age-14)/15/100);
if k_{sd-m}≤(d₂+(age-14)/15/100), then a₂=0;
wherein if |k_{sd-m-0}−k_{sd-m-ts}|≥40 and (k_{sd-m-0}+k_{sd-m-ts})/2≥k_{sd-m-2}, then k_{sd-m}=2×k_{sd-m-2}−(k_{sd-m-0}+k_{sd-m-ts})/2, otherwise k_{sd-m}=k_{sd-m-2};
$${k}_{sd - m - 0} = \frac{{t}_{s} {h}_{sd}}{{\int }_{0}^{{t}_{s}} h dt} , {k}_{sd - m - ts} = \frac{{t}_{s} {h}_{sd}}{{\int }_{0}^{2 {t}_{s}} h dt} ,$$
age is age, and d₂=1.17 to 1.27.

16. The method for correcting pulse wave transit time associated with systolic blood pressure according to claim 11, characterized in that, the third correction variable a₃ is calculated by the following formulas:
if c₄<k_{d-m-a}<c₅, then a₃=0;
if k_{sd-m-0}<d₆ or k_{sd-m-2}>d₇, then a₃=0;
if k_{sd-m-0}≥d₆+0.10 and k_{sd-m-2}≤d₈ and k_{d-m-a}≤c₄, then a₃=(c₄−k_{d-m-a})×67/100;
if
$$\{ \begin{matrix}{d}_{6} \leq {k}_{sd - m - 0} < {d}_{6} + 0.1 \\ {k}_{d - m - a} \leq {c}_{4}\end{matrix} or \{ \begin{matrix}{d}_{8} < {k}_{sd - m - 2} < {d}_{7} \\ {k}_{d - m - a} \leq {c}_{4}\end{matrix} ,$$
then a₃=(c₄₋₁k_{d-m-a})×50/100;
if k_{sd-m-0}≥d₆+0.10 and k_{sd-m-2}≤d₈ and k_{d-m-a}≥c₅, then a₃=(c₅−k_{d-m-a})×62/100;
if
$$\{ \begin{matrix}{d}_{6} \leq {k}_{sd - m - 0} < {d}_{6} + 0.1 \\ {k}_{d - m - a} \geq {c}_{5}\end{matrix} or \{ \begin{matrix}{d}_{8} < {k}_{sd - m - 2} < {d}_{7} \\ {k}_{d - m - a} \geq {c}_{5}\end{matrix} ,$$
then a₃=(c₅k_{d-m-a})×45/100;
wherein if |k^{sd-m-0}−k_{sd-m-ts}|≥40 and (k_{sd-m-0}+k_{sd-m-ts})/2≥k_{sd-m-2} and k_{sd-m-ts}≥d₃₋₂, then k_{d-m-a}=(k_{d-m-t}_{_{d}}₋₁+k_{d-m-t}_{_{d-toe}}+(k_{sd-m-0}+k_{sd-m-ts})/2−k_{sd-m-2})/2, otherwise k_{d-m-a}=(k_{d-m-t}_{_{d}}₋₁+k_{d-m-t}_{_{d-toe}})/2;
if k_{sd-m-ts}≤d₃₋₂, then k_{d-m-t}_{_{d}}₋₁=k_{d-m-t}_{_{d}}−(d₃₋₂−k_{sd-m-ts})×75/100; if k_{d-m-t}_{_{d}}≤d₃, then k_{d-m-t}_{_{d}}₋₁=d₃; if k_{d-m-t}_{_{d-toe}}≤d₃, then k_{d-m-t}_{_{d-toe}}=d₃;
$${k}_{d - m - {t}_{d}} = \frac{{\int }_{{t}_{s}}^{{t}_{s} + {t}_{d}} hdt}{{t}_{d} {h}_{max}} , {k}_{sd - m - 0} = \frac{{t}_{s} {h}_{sd}}{{\int }_{0}^{{t}_{s}} hdt} , {k}_{sd - m - 2} = \frac{2 {t}_{s} {h}_{sd}}{{\int }_{0}^{2 {t}_{s}} hdt} ,$$
$${k}_{sd - m - ts} = \frac{{t}_{s} {h}_{sd}}{{\int }_{{t}_{s}}^{2 {t}_{s}} hdt} , {k}_{d - m - {t}_{d - toe}} = \frac{{\int }_{{t}_{s - toe}}^{{t}_{s - toe} + {t}_{d - toe}} hdt}{{t}_{d - toe} {h}_{max - toe}} ;$$
and
c₄=(d₄+(age-14)/8)/100, d₄=23 to 35, c₅=(d₅+(age-14)/8)/100, d₅=27 to 39, d₆=0.97 to 1.03, d₇=1.52 to 1.58, d₈=1.42 to 1.48, d₃₋₂=1.21 to 1.31, d₃=0.02 to 0.14, and age is age.

17. The method for correcting pulse wave transit time associated with systolic blood pressure according to claim 11, characterized in that, the fourth correction variable a4 is calculated by the following formulas: if kₛ₋ₜ₋ₜₒₑ>0.8, then a₄=kₛ₋ₜ₋ₜₒₑ-0.8;
if kₛ₋ₜ₋ₜₒₑ≤0.8, then a₄=0;
wherein if tₘₐₓ₋ₜₒₑ≥t_{ch-toe}, then
$${k}_{s - t - toe} = \frac{{t}_{max - toe} + {t}_{ch - toe} + 400}{( {t}_{s - toe} + 200 ) \times 2} ,$$
otherwise
$${k}_{s - t - toe} = \frac{{t}_{max - toe} + 200}{{t}_{s - toe} + 200} .$$

18. The method for correcting pulse wave transit time associated with systolic blood pressure according to claim 11, characterized in that, the fifth correction variable as is calculated by the following formulas:
if kₛ₋ₘ₋ₜₒₑ<d₉, then a₅=0;
if kₛ₋ₘ₋ₜₒₑ≥d₉ and kₛ₋ₜ₋ₜₒₑ≥0.8, then a₅=kₛ₋ₘ₋ₜₒₑ−d₉;
if kₛ₋ₘ₋ₜₒₑ≥d₉ and kₛ₋ₜ₋ₜₒₑ<0.8, then a₅=(kₛ₋ₘ₋ₜₒₑ−d₉)/2;
wherein d₉=0.67 to 0.73,
$${k}_{s - m - toe} = \frac{{\int }_{0}^{{t}_{s - toe}} h dt}{{t}_{s - toe} {h}_{max - toe}} .$$

19. The method for correcting pulse wave transit time associated with systolic blood pressure according to claim 11, characterized in that, the sixth correction variable a₆ is calculated by the following formulas:
if kₛ₋ₘ₋ₜₒₑ₋ₑₐᵣ<1.0, then a₆=0;
when kₛ₋ₘ₋ₜₒₑ₋ₑₐᵣ>1.08, then c₆=1.08, meantime, if tₛ>220 and k_{sd-m-0}>0.88, then a₆=c₆−1.0, if tₛ<160 or k_{sd-m-0}<0.80, then a₆=(c₆−1.0)×0.34, if 160<tₛ≤220 or 0.80<k_{sd-m-0}≤0.88, then a₆=(c₆−1.0)×0.67;
when 1.0≤kₛ₋ₘ₋ₜₒₑ₋ₑₐᵣ≤1.08, then c₆=kₛ₋ₘ₋ₜₒₑ₋ₑₐᵣ−1.0, meantime, if tₛ>220 and k_{sd-m-0}>0.88, then a₆=c₆, if tₛ≤160 or k_{sd-m-0}≤0.80, then a₆=c₆×0.34, if 160<tₛ≤220 or 0.80<k_{sd-m-0}≤0.88, then a₆=c₆×0.67;
wherein
$${k}_{s - m - toe - ear} = \frac{{h}_{max} {\int }_{0}^{{t}_{s - toe}} h dt + ( {t}_{s} + {t}_{s - toe} ) \times 100}{{h}_{max - toe} {\int }_{0}^{{t}_{s}} h dt + ( {t}_{s} + {t}_{s - toe} ) \times 100} ,$$
$${k}_{sd - m - 0} = \frac{{t}_{s} {h}_{sd}}{{\int }_{0}^{{t}_{s}} h dt} .$$

20. The method for correcting pulse wave transit time associated with systolic blood pressure according to claim 11, characterized in that, the seventh correction variable a₇ is calculated by the following formulas:
if kₜₛ₋ₜₒₑ₋ₑₐᵣ<1.0, then a₇=0;
when kₜₛ₋ₜₒₑ₋ₑₐᵣ>1.08, then c₇=1.08, meantime, if tₛ>220 and k_{sd-m-0}>0.88, then a₇=c₇−1.0, if tₛ<160 or k_{sd-m-0}<0.80, then a₇=(c₇−1.0)×0.34, if 160<tₛ≤220 or 0.80<k_{sd-m-0}≤0.88, then a₇=(c₇−1.0)×0.67;
when 1.0≤kₜₛ₋ₜₒₑ₋ₑₐᵣ≤1.08, then c₇=kₜₛ₋ₜₒₑ₋ₑₐᵣ−1.0, meantime, if tₛ>220 and k_{sd-m-0}>0.88, then a₇=c₇, if tₛ≤160 or k_{sd-m-0}≤0.80, then a₇=c₇×0.34, if 160<tₛ≤220 or 0.80<k_{sd-m-0}≤0.88, then c₇=c₇×0.67;
wherein
$${k}_{ts - toe - ear} = \frac{{t}_{s - toe} + 825}{{t}_{s} + 825} , {k}_{sd - m - 0} = \frac{{t}_{s} {h}_{sd}}{{\int }_{0}^{{t}_{s}} h dt} .$$
